SEQUENCE LISTING (4 sequences)

SEQ ID NO: 1 - protein, 303 aa, Artificial Sequence
  circularly permuted interleukin-2 (IL-2) fused to the extracellular portion of an IL-2Ralpha chain
SKNFHLRPRDLISNINVIVLELKGSETTFMCEYADETATIVEFLNRWITFSQSIISTLTGGSSSTKKTQLQLEHLLLDLQMILNGINNYKNPKLTRMLTFKFYMPKKATELKHLQCLEEELKPLEEVLNLAQGSGGGSELCDDDPPEIPHATFKAMAYKEGTMLNCECKRGFRRIKSGSLYMLCTGNSSHSSWDNQCQCTSSATRNTTKQVTPQPEEQKERKTTEMQSPMQPVDQASLPGHCREPPPWENEATERIYHFVVGQMVYYQCVQGYRALHRGPAESVCKMTHGKTRWTQPQLICTG

SEQ ID NO: 2 - protein, 29 aa, Artificial Sequence
  Residue 177-205
SGSLYMLCTGNSSHSSWDNQCQCTSSATR

SEQ ID NO: 3 - protein, 4 aa, Artificial Sequence
  Residue 206-209
NTTK

SEQ ID NO: 4 - protein, 12 aa, Artificial Sequence
  Resiude 210-221
QVTPQPEEQKER

(19) United States
(12) Patent Application Publication
(10) Pub. No.: US 2021/0253631 A1
(43) Pub. Date: Aug. 19, 2021

(54) METHODS OF PURIFICATION

(71) Applicant: Alkermes, Inc., Waltham, MA (US)

(72) Inventors: R. Blair McNeill, Apex, NC (US); Raj Prabu Vijayakumar Saraswathi, Westchester, OH (US); Matthew J. Pete, Lebanon, OH (US)

(21) Appl. No.: 17/154,108

(22) Filed: Jan. 21, 2021

Related U.S. Application Data
(60) Provisional application No. 62/965,578, filed on Jan. 24, 2020.

(51) Int. Cl. C07K 1/16; C07K 1/18; C07K 1/22; C07K 14/715

(52) U.S. Cl. CPC C07K 1/165; C07K 14/7155; C07K 1/22; C07K 1/18

(57) ABSTRACT

Provided herein are methods of purifying polypeptides comprising a circularly permuted interleukin-2 (IL-2) fused to the extracellular portion of an IL-2Rα chain.

### RELATED APPLICATIONS

This application claims the benefit of U.S. Provisional Application No. 62/965,578, filed Jan. 24, 2020, the entire disclosure of which is hereby incorporated by reference.

### SEQUENCE LISTING

This application contains a sequence listing which has been submitted electronically in ASCII format and is hereby incorporated by reference in its entirety (said ASCII copy, created on Jan. 21, 2021, is named “713999 ALW-028 ST25.txt” and is 3,711 bytes in size).

### FIELD OF THE INVENTION

This disclosure relates to methods of purifying polypeptides comprising a circularly permuted interleukin-2 (IL-2) fused to the extracellular portion of an IL-2Rα chain.

### BACKGROUND

Polypeptides comprising a circularly permuted interleukin-2 (IL-2) fused to the extracellular portion of an IL-2Rα chain interleukin-2 (IL-2) interleukin-2 receptor alpha (IL-2Rα) hold great promise as anti-cancer agents. These polypeptides retain full ability to signal through the intermediate-affinity IL-2R complex that is expressed on memory CD8+ T cells and Natural Killer (NK) cells, but are sterically prevented from binding to the high-affinity IL-2R complex that is preferentially expressed on CD4+ FOXP3+ regulatory T cells (CD4+ Tregs) and endothelial cells. As a result of this selective IL-2R binding, the fusion proteins selectively activate CD8+ T cells and NK cells, thereby promoting tumor cell killing. The inability to activate the high-affinity IL-2R on endothelial cells may also reduce the risk of toxicity due to capillary leak syndrome, a known risk of IL-2 therapies.

In order to be used as therapeutics, the aforementioned polypeptides must first be separated from other biomolecule contaminants that are present during manufacture. Accordingly, there is a need in the art for methods of purifying such polypeptides.

### SUMMARY

The present disclosure provides methods of purifying polypeptides comprising a circularly permuted IL-2 fused to the extracellular portion of an IL-2Rα chain.

Accordingly, in one aspect, the disclosure provides a method of purifying a polypeptide comprising a circularly permuted IL-2 fused to the extracellular portion of an IL-2Rα chain, the method comprising: a. contacting a clarified cell supernatant comprising the polypeptide and host cell protein (HCP) with a first chromatography matrix under conditions such that the polypeptide binds to the matrix, and selectively eluting the polypeptide from the matrix in a first eluate; b. adjusting the pH of the first eluate from step (a) to at least 10.5 and at most 14.0 (e.g., about 10.5, 10.6, 10.7, 10.8, 10.9, 11.1, 11.2, 11.3, 11.4, 11.5, 11.6, 11.7, 11.8, 11.9, 12.0, 12.1, 12.2, 12.3, 12.4, 12.5, 12.6, 12.7, 12.8, 12.9, 13.0, 13.1, 13.2, 13.3, 13.4, 13.5, 13.6, 13.7, 13.8, 13.9, or 14.0) to produce a pH-adjusted eluate; and c. contacting the pH-adjusted eluate from step (b) with a second chromatography matrix such that the polypeptide binds to the matrix, and selectively eluting the polypeptide from the matrix in a second eluate, thereby purifying the polypeptide.

In certain embodiments, the polypeptide comprises an amino acid sequence that is at least 85%, 90%, 95%, or 99% identical to of SEQ ID NO: 1.

In certain embodiments, the polypeptide comprises the amino acid sequence of SEQ ID NO: 1.

In certain embodiments, the first chromatography matrix comprises an anion exchange chromatography (AEX) matrix.

In certain embodiments, prior to contact with the AEX matrix the clarified cell supernatant is buffer-exchanged into a solution having a conductivity of about 1-2 mS/cm and a pH of about 8.0-8.5.

In certain embodiments, the AEX matrix comprises quaternary amine groups.

In certain embodiments, the AEX matrix comprises hydroxylated methacrylic polymer beads linked functionalized with quaternary amine groups.

In certain embodiments, the mean diameter of the beads is about 75 μm. In certain embodiments, the mean pore size of the beads is about 100 mu.

In certain embodiments, the polypeptide is eluted from the AEX matrix at a salt concentration equivalent of conductivity of about 15 to about 25 mS/cm.

In certain embodiments, the polypeptide is eluted from the AEX matrix at a salt concentration equivalent of conductivity of about 20 to about 25 mS/cm.

In certain embodiments, the polypeptide is eluted from the AEX matrix at a salt concentration equivalent of conductivity of about 15 mS/cm, about 16 mS/cm, about 17 mS/cm, about 18 mS/cm, about 19 mS/cm, about 20 mS/cm, about 21 mS/cm, about 22 mS/cm, about 23 mS/cm, about 24 mS/cm, about 25 mS/cm.

In certain embodiments, the polypeptide is eluted from the AEX matrix using a solution of about 0.20-0.25 M sodium chloride at about pH 8.4-8.6.

In certain embodiments, the pH of the first eluate is adjusted (e.g., increased) using sodium carbonate and/or sodium hydroxide.

In certain embodiments, the pH of the first eluate is adjusted (e.g., increased) using sodium carbonate or sodium hydroxide at a ratio of about 0.1 kg sodium carbonate or sodium hydroxide to about 1 kg of the first eluate.

In certain embodiments, the pH of the pH-adjusted eluate is maintained at or above 10.5 but below 12.0 (e.g., at or above 10.7 but below 11.0 (e.g., about 10.7, 10.8, or 10.9) for at least 30 mins (e.g., at least 1 hour).

In certain embodiments, the pH of the pH-adjusted eluate is maintained at or above 10.5 but below 12.0 (e.g., at or above 10.7 but below 11.0 (e.g., about 10.7, 10.8, or 10.9) for at least 30 mins (e.g., at least 1 hour), wherein the pH at or above 10.5 but below 12.0 (e.g., at or above 10.7 but below 11.0 (e.g., about 10.7, 10.8, or 10.9) is achieved by adding sodium carbonate or sodium hydroxide at a ratio of about 0.1 kg sodium carbonate or sodium hydroxide to about 1 kg of the first eluate.

In certain embodiments, the method further comprises lowering the pH of the pH-adjusted eluate by adding citric acid (e.g., prior to contacting the pH-adjusted eluate with the second chromatography matrix).

In certain embodiments, method further comprises lowering the pH of the pH-adjusted eluate to about pH 8.3-8.7 (e.g., prior to contacting the pH-adjusted eluate with the second chromatography matrix).

In certain embodiments, prior to contact with the second chromatography matrix the conductivity of the pH-adjusted eluate is altered to be about 130 to about 160 mS/cm and the pH is altered to be about 8.5±0.2.

In certain embodiments, prior to contact with the second chromatography matrix the conductivity of the pH-adjusted eluate is altered to be about 130 mS/cm, about 131 mS/cm, about 132 mS/cm, about 133 mS/cm, about 134 mS/cm, about 135 mS/cm, about 136 mS/cm, about 137 mS/cm, about 138 mS/cm, about 139 mS/cm, about 140 mS/cm, about 141 mS/cm, about 142 mS/cm, about 143 mS/cm, about 144 mS/cm, about 145 mS/cm, about 146 mS/cm, about 147 mS/cm, about 148 mS/cm, about 149 mS/cm, about 150 mS/cm, about 151 mS/cm, about 152 mS/cm, about 153 mS/cm, about 154 mS/cm, about 155 mS/cm, about 156 mS/cm, about 157 mS/cm, about 158 mS/cm, about 159 mS/cm, or about 160 mS/cm and the pH is altered to be about 8.5±0.2.

In certain embodiments, prior to contact with the second chromatography matrix the conductivity of the pH-adjusted eluate is altered to be about 134 to about 160 mS/cm and the pH is altered to be about 8.5±0.2.

In certain embodiments, prior to contact with the second chromatography matrix the conductivity of the pH-adjusted eluate is altered to be about 155 to about 160 mS/cm and the pH is altered to be about 8.5±0.2.

In certain embodiments, the conductivity of the pH-adjusted eluate is altered using ammonium sulfate.

In certain embodiments, the second chromatography matrix comprises a hydrophobic interaction chromatography (HIC) matrix.

In certain embodiments, prior to contact with the HIC matrix the pH-adjusted eluate is altered to comprise about 1-1.2 M ammonium sulfate.

In certain embodiments, the HIC matrix comprises polypropylene glycol groups.

In certain embodiments, the HIC matrix comprises hydroxylated methacrylic polymer beads linked to polypropylene glycol groups.

In certain embodiments, the mean diameter of the beads is about 40 to about 90 μm.

In certain embodiments, the mean pore size of the beads is about 75 nm.

In certain embodiments, the polypeptide is eluted from the HIC matrix using a salt concentration equivalent of conductivity of about 100 to about 140 mS/cm.

In certain embodiments, the polypeptide is eluted from the HIC matrix using a salt concentration equivalent of conductivity of about 125 to about 140 mS/cm.

In certain embodiments, the polypeptide is eluted from the HIC matrix using a sequential multistep gradient of decreasing salt concentration.

In certain embodiments, the polypeptide is eluted from the HIC matrix using a buffer comprising about 0.85 to about 0.95 M ammonium sulfate at pH 8.5±0.2.

In certain embodiments, prior to contact with the HIC matrix the pH-adjusted eluate is filtered through a 0.2 μm filter.

In certain embodiments, prior to contact with the HIC matrix the pH-adjusted eluate is subject to viral inactivation.

In certain embodiments, the viral inactivation is achieved by admixture of the pH-adjusted eluate with tri-n-butyl phosphate and polysorbate 20.

In certain embodiments, the polypeptide is further purified from the second eluate using mixed-mode chromatography (MMC).

In certain embodiments, the polypeptide is further purified from the second eluate using MMC followed by AEX.

In certain embodiments, the clarified cell supernatant is from a Chinese hamster ovary (CHO) cell culture.

In certain embodiments, the polypeptide in the second eluate is at least 90% pure. In certain embodiments, purity of the polypeptide is determined by Reverse Phase (RP) HPLC.

In another aspect, the disclosure provides a method for reducing host cell protein (HCP) content from a clarified cell supernatant containing a polypeptide comprising a circularly permuted IL-2 fused to the extracellular portion of an IL-2Rα chain, the method comprising contacting a partially purified polypeptide with an AEX matrix under conditions such that the polypeptide binds to the AEX matrix, and selectively eluting the polypeptide from the AEX matrix under gradient elution conditions, thereby reducing HCP content from the polypeptide.

In certain embodiments, the polypeptide comprises: an amino acid sequence that is at least 85%, 90%, 95%, or 99% identical to SEQ ID NO: 1; or that comprises the amino acid sequence of SEQ ID NO: 1.

In certain embodiments, the method further comprises contacting the clarified cell supernatant comprising the polypeptide and HCP with one or more chromatography resins to obtain the partially purified polypeptide.

In certain embodiments, the HCP content is ≥ about 300 ppm prior to contacting the partially purified polypeptide with the AEX matrix. In certain embodiments, the HCP content is ≥ about 150 ppm prior to contacting the partially purified polypeptide with the AEX matrix.

In certain embodiments, the HCP content is ≤ about 100 ppm after eluting the polypeptide from the AEX matrix under gradient elution conditions. In certain embodiments, the HCP content is ≤ about 50 ppm after eluting the polypeptide from the AEX matrix under gradient elution conditions. In certain embodiments, the HCP content is ≤ about 25 ppm after eluting the polypeptide from the AEX matrix under gradient elution conditions.

In certain embodiments, the gradient elution conditions comprise one or more of: increasing the conductivity of an elution buffer over time; increasing the salt concentration of an elution buffer over time; or decreasing the pH of an elution buffer over time.

In certain embodiments, the conductivity of the elution buffer is increased from about ≤5 mS/cm to about ≥15 mS/cm. In certain embodiments, the conductivity of the elution buffer is increased from about ≤2 mS/cm to about ≥15 mS/cm. In certain embodiments, the conductivity of the elution buffer is increased from about ≤2 mS/cm to about ≥20 mS/cm. In certain embodiments, the conductivity of the elution buffer is increased to between about 15 mS/cm to about 100 mS/cm. In certain embodiments, the conductivity of the elution buffer is increased to between about 20 mS/cm to about 50 mS/cm. In certain embodiments, the elution buffer comprises a final conductivity of between about 20 mS/cm to about 50 mS/cm. In certain embodiments, the elution buffer initially comprises a conductivity of about ≤5 mS/cm. In certain embodiments, the elution buffer initially comprises a conductivity of about ≤2 mS/cm.

In certain embodiments, the salt concentration of the elution buffer is increased from about 0 M salt to about 1.5 M salt. In certain embodiments, the salt concentration of the elution buffer is increased from about 0 M salt to about 1.0 M salt. In certain embodiments, the salt concentration of the elution buffer is increased from about 0 M salt to about 0.5 M salt. In certain embodiments, the elution buffer comprises a final salt concentration of between about 0.2 M salt to about 1.0 M salt. In certain embodiments, the elution buffer comprises a final salt concentration of about 0.2 M salt. In certain embodiments, the salt comprises sodium chloride.

In certain embodiments, the elution buffer further comprises a pH of about 7.0 to about 9.0. In certain embodiments, the elution buffer further comprises a pH of about 8.0.

In certain embodiments, the one or more chromatography resins to obtain the partially purified polypeptide are selected from the group consisting of AEX, HIC, and MMC.

In certain embodiments, obtaining the partially purified polypeptide comprises the steps of: a1) contacting the clarified cell supernatant comprising the polypeptide and HCP with a first AEX matrix under conditions such that the polypeptide binds to the AEX matrix, and selectively eluting the polypeptide from the AEX matrix in a first eluate; a2) contacting the first eluate from step (a1) with a HIC matrix such that the polypeptide binds to the HIC matrix, and selectively eluting the polypeptide from the HIC matrix in a second eluate; and a3) contacting the second eluate from step (a2) with a MMC matrix such that the polypeptide binds to the MMC matrix, and selectively eluting the polypeptide from the MMC matrix in a third eluate, thereby obtaining the partially purified polypeptide.

In certain embodiments, prior to contact with the first AEX matrix the clarified cell supernatant is buffer-exchanged into a solution having a conductivity of about 1-2 mS/cm and a pH of about 8.0-8.5.

In certain embodiments, the first AEX matrix comprises quaternary amine groups. In certain embodiments, the first AEX matrix comprises hydroxylated methacrylic polymer beads functionalized with quaternary amine groups. In certain embodiments, the mean diameter of the beads is about 75 μm. In certain embodiments, the mean pore size of the beads is about 100 nm.

In certain embodiments, the polypeptide is eluted from the first AEX matrix using a solution having a salt concentration equivalent of conductivity of about 15 to about 25 mS/cm.

In certain embodiments, the polypeptide is eluted from the first AEX matrix using a solution having a salt concentration equivalent of conductivity of about 20 to about 25 mS/cm.

In certain embodiments, the polypeptide is eluted from the AEX matrix at a salt concentration equivalent of conductivity of about 15 mS/cm, about 16 mS/cm, about 17 mS/cm, about 18 mS/cm, about 19 mS/cm, about 20 mS/cm, about 21 mS/cm, about 22 mS/cm, about 23 mS/cm, about 24 mS/cm, about 25 mS/cm.

In certain embodiments, the polypeptide is eluted from the first AEX matrix using an aqueous solution of about 0.20-0.25 M sodium chloride at about pH 8.4-8.6.

In certain embodiments, the pH of the first eluate is adjusted (e.g., increased) using sodium carbonate and/or sodium hydroxide to produce a pH-adjusted first eluate.

In certain embodiments, the pH of the first eluate is adjusted (e.g., increased) using sodium carbonate or sodium hydroxide at a ratio of about 0.1 kg sodium carbonate or sodium hydroxide to about 1 kg of the first eluate.

In certain embodiments, the pH of the pH-adjusted eluate is maintained at or above 10.5 but below 12.0 (e.g., at or above 10.7 but below 11.0 (e.g., about 10.7, 10.8, or 10.9) for at least 30 mins (e.g., at least 1 hour).

In certain embodiments, the pH of the pH-adjusted eluate is maintained at or above 10.5 but below 12.0 (e.g., at or above 10.7 but below 11.0 (e.g., about 10.7, 10.8, or 10.9) for at least 30 mins (e.g., at least 1 hour), wherein the pH at or above 10.5 but below 12.0 (e.g., at or above 10.7 but below 11.0 (e.g., about 10.7, 10.8, or 10.9) is achieved by adding sodium carbonate or sodium hydroxide at a ratio of about 0.1 kg sodium carbonate or sodium hydroxide to about 1 kg of the first eluate.

In certain embodiments, the method further comprises lowering the pH of the pH-adjusted first eluate by adding citric acid. In certain embodiments, the method further comprises lowering the pH of the pH-adjusted first eluate to about pH 8.3-8.7.

In certain embodiments, prior to contact with the HIC matrix the conductivity of the pH-adjusted first eluate is altered to be about 155 to about 160 mS/cm and the pH is altered to be about 8.5±0.2.

In certain embodiments, the conductivity of the pH-adjusted first eluate is altered using ammonium sulfate. In certain embodiments, prior to contact with the HIC matrix the pH-adjusted first eluate is altered to comprise about 1-1.2 M ammonium sulfate.

In certain embodiments, the HIC matrix comprises polypropylene glycol groups. In certain embodiments, the HIC matrix comprises hydroxylated methacrylic polymer beads linked to polypropylene glycol groups. In certain embodiments, the mean diameter of the beads is about 40 to about 90 μm. In certain embodiments, the mean pore size of the beads is about 75 nm.

In certain embodiments, the polypeptide is eluted from the HIC matrix using a solution having a salt concentration equivalent of conductivity of about 100 to about 140 mS/cm.

In certain embodiments, the polypeptide is eluted from the HIC matrix using a solution having a salt concentration equivalent of conductivity of about 125 to about 140 mS/cm.

In certain embodiments, the polypeptide is eluted from the HIC matrix using a sequential multistep gradient of decreasing salt concentration.

In certain embodiments, the polypeptide is eluted from the HIC matrix using a buffer comprising about 0.85 to about 0.95 M ammonium sulfate at pH 8.5±0.2.

In certain embodiments, prior to contact with the HIC matrix the pH-adjusted eluate is filtered through a 0.2 μm filter.

In certain embodiments, prior to contact with the HIC matrix the pH-adjusted first eluate is subject to viral inactivation.

In certain embodiments, the viral inactivation is achieved by admixture of the pH-adjusted eluate with tri-n-butyl phosphate and polysorbate 20.

In certain embodiments, the clarified cell supernatant is from a Chinese hamster ovary (CHO) cell culture.

In one aspect, the disclosure provides a method for reducing host cell protein (HCP) content from a clarified cell supernatant to containing a polypeptide comprising a circularly permuted IL-2 fused to the extracellular portion of an IL-2Rα chain, the method comprising the steps of: contacting the clarified cell supernatant comprising the polypeptide and HCP with a first AEX matrix under conditions such that the polypeptide binds to the first AEX matrix, and selectively eluting the polypeptide from the first AEX matrix in a first eluate; contacting the first eluate with a HIC matrix such that the polypeptide binds to the HIC matrix, and selectively eluting the polypeptide from the HIC matrix in a second eluate; contacting the second eluate with a MMC matrix such that the polypeptide binds to the MMC matrix, and selectively eluting the polypeptide from the MMC matrix in a third eluate; and contacting the third eluate with a second AEX matrix under conditions such that the polypeptide binds to the second AEX matrix, and selectively eluting the polypeptide from the second AEX matrix under gradient elution conditions, thereby reducing HCP content from the polypeptide, wherein the HCP content is ≤ about 50 ppm after step (d).

In certain embodiments, the polypeptide comprises: an amino acid sequence that is at least 85%, 90%, 95%, or 99% identical to SEQ ID NO: 1; or that comprises the amino acid sequence of SEQ ID NO: 1.

In certain embodiments, an affinity purification step is not used.

In one aspect, the disclosure provides a composition comprising a polypeptide comprising a circularly permuted IL-2 fused to the extracellular portion of an IL-2Rα chain, wherein the HCP content of the composition comprises ≤ about 100 ppm.

In certain embodiments, the HCP content of the composition comprises ≤ about 50 ppm.

In certain embodiments, the polypeptide comprises: an amino acid sequence that is at least 85%, 90%, 95%, or 99% identical to SEQ ID NO: 1; or that comprises the amino acid sequence of SEQ ID NO: 1.

In certain embodiments, the composition is produced by the method recited above.

In one aspect, the disclosure provides a method of improving the serum half-life of a composition comprising a plurality of polypeptides, each polypeptide of the plurality comprising a circularly permuted IL-2 fused to the extracellular portion of an IL-2Rα chain, the method comprising the steps of: contacting the clarified cell supernatant comprising the polypeptide with a first AEX matrix under conditions such that the polypeptide binds to the first AEX matrix, and selectively eluting the polypeptide from the first AEX matrix in a first eluate; contacting the first eluate with a HIC matrix such that the polypeptide binds to the HIC matrix, and selectively eluting the polypeptide from the HIC matrix in a second eluate; contacting the second eluate with a MMC matrix such that the polypeptide binds to the MMC matrix, and selectively eluting the polypeptide from the MMC matrix in a third eluate; and contacting the third eluate with a second AEX matrix under conditions such that the polypeptide binds to the second AEX matrix, and selectively eluting the polypeptide from the second AEX, thereby improving the serum half-life of the composition.

In certain embodiments, the polypeptide comprises: an amino acid sequence that is at least 85%, 90%, 95%, or 99% identical to SEQ ID NO: 1; or that comprises the amino acid sequence of SEQ ID NO: 1.

In certain embodiments, the method further comprises adjusting the pH of the first eluate from step (a) to at least 10.5 and at most 11.5 before contacting the first eluate with the HIC matrix of step (b).

In one aspect, the disclosure provides a composition comprising a plurality of polypeptides, each polypeptide of the plurality comprising the amino sequence of SEQ ID NO: 1 linked to one or more glycan species, wherein the one or more glycan species are linked to the polypeptide at one or more of amino acid positions N187, N206, and T212 of SEQ ID NO: 1.

In certain embodiments, the glycan species at amino acid position N187 of SEQ ID NO: 1 are selected from the group consisting of: Hex5HexNAc4FucNeuAc2; Hex6HexNAc5FucNeuAc2; Hex5HexNAc4FucNeuAc; Hex6HexNAc5FucNeuAc3; Hex4HexNAc4FucNeuAc; Hex5HexNAc5NeuAc2; Hex5HexNAc4Fuc; Hex3HexNAc4Fuc; Hex4HexNAc4Fuc; Hex6HexNAc5Fuc; and Hex5HexNAc5Fuc; wherein Hex represents hexose, HexNAc represents N-acetylhexosamine, NeuAc represents N-acetylneuraminic acid, Fuc represents fucose, and the number represents the number of each glycan structure.

In certain embodiments, the glycan species at amino acid position N206 of SEQ ID NO: 1 are selected from the group consisting of: Hex6HexNAc5FucNeuAc3; Hex5HexNAc4FucNeuAc2; Hex6HexNAc5FucNeuAc2; Hex7HexNAc6FucNeuAc3; Hex6HexNAc5FucNeuAc; Hex5HexNAc4FucNeuAc; Hex5HexNAc4Fuc; and Hex4HexNAc4Fuc; wherein Hex represents hexose, HexNAc represents N-acetylhexosamine, NeuAc represents N-acetylneuraminic acid, Fuc represents fucose, and the number represents the number of each glycan structure.

In certain embodiments, the glycan species at amino acid position T212 of SEQ ID NO: 1 are selected from the group consisting of: HexHexNAc; HexHexNAcNeuAc; and HexHexNAcNeuAc2; wherein Hex represents hexose, HexNAc represents N-acetylhexosamine, NeuAc represents N-acetylneuraminic acid, and the number represents the number of each glycan structure.

In certain embodiments, the overall percent of glycan species at amino acid position N187 of SEQ ID NO: 1 of the plurality of polypeptides in the composition comprises: about 60% to about 70% Hex5HexNAc4FucNeuAc2; about 4% to about 6% Hex6HexNAc5FucNeuAc2; about 7% to about 10% Hex5HexNAc4FucNeuAc; about 15% to about 17% Hex6HexNAc5FucNeuAc3; and about 3% to about 4% Hex5HexNAc5NeuAc2; wherein Hex represents hexose, HexNAc represents N-acetylhexosamine, NeuAc represents N-acetylneuraminic acid, Fuc represents fucose, and the number represents the number of each glycan structure.

In certain embodiments, the overall percent of glycan species at amino acid position N187 of SEQ ID NO: 1 of the plurality of polypeptides in the composition comprises: about 60% to about 70% Hex5HexNAc4FucNeuAc2; about 4% to about 6% Hex6HexNAc5FucNeuAc2; about 7% to about 10% Hex5HexNAc4FucNeuAc; about 15% to about 17% Hex6HexNAc5FucNeuAc3; about 0.5% to about 1.5% Hex4HexNAc4FucNeuAc; about 3% to about 4% Hex5HexNAc5NeuAc2; about 0% to about 0.5% Hex5HexNAc4Fuc; about 0% to about 0.5% Hex3HexNAc4Fuc; about 0% to about 0.5% Hex4HexNAc4Fuc; about 0% to about 0.5% Hex6HexNAc5Fuc; and about 0% to about 0.5% Hex5HexNAc5Fuc; wherein Hex represents hexose, HexNAc represents N-acetylhexosamine, NeuAc represents N-acetylneuraminic acid, Fuc represents fucose, and the number represents the number of each glycan structure.

In certain embodiments, the overall percent of glycan species at amino acid position N206 of SEQ ID NO: 1 of the plurality of polypeptides in the composition comprises: about 3% to about 5% Hex6HexNAc5FucNeuAc3; about 75% to about 85% Hex5HexNAc4FucNeuAc2; about 2% to about 4% Hex6HexNAc5FucNeuAc2; about 5% to about 12% Hex5HexNAc4FucNeuAc; about 1% to about 3% Hex5HexNAc4Fuc; and wherein Hex represents hexose, HexNAc represents N-acetylhexosamine, NeuAc represents N-acetylneuraminic acid, Fuc represents fucose, and the number represents the number of each glycan structure.

In certain embodiments, the overall percent of glycan species at amino acid position N206 of SEQ ID NO: 1 of the plurality of polypeptides in the composition comprises: about 3% to about 5% Hex6HexNAc5FucNeuAc3; about 75% to about 85% Hex5HexNAc4FucNeuAc2; about 2% to about 4% Hex6HexNAc5FucNeuAc2; about 0.5% to about 1.5% Hex7HexNAc6FucNeuAc3; about 0% to about 1% Hex6HexNAc5FucNeuAc; about 5% to about 12% Hex5HexNAc4FucNeuAc; about 1% to about 3% Hex5HexNAc4Fuc; and about 0.5% to about 2% Hex4HexNAc4Fuc; wherein Hex represents hexose, HexNAc represents N-acetylhexosamine, NeuAc represents N-acetylneuraminic acid, Fuc represents fucose, and the number represents the number of each glycan structure.

In certain embodiments, the overall percent of glycan species at amino acid position T212 of SEQ ID NO: 1 of the plurality of polypeptides in the composition comprises: about 14% to about 18% HexHexNAcNeuAc; and about 8% to about 13% HexHexNAcNeuAc2; wherein Hex represents hexose, HexNAc represents N-acetylhexosamine, NeuAc represents N-acetylneuraminic acid, and the number represents the number of each glycan structure.

In certain embodiments, the overall percent of glycan species at amino acid position T212 of SEQ ID NO: 1 of the plurality of polypeptides in the composition comprises: about 0% to about 1% HexHexNAc; about 14% to about 18% HexHexNAcNeuAc; and about 8% to about 13% HexHexNAcNeuAc2; wherein Hex represents hexose, HexNAc represents N-acetylhexosamine, NeuAc represents N-acetylneuraminic acid, and the number represents the number of each glycan structure.

In one aspect, the disclosure provides a composition comprising a plurality of polypeptides, each polypeptide of the plurality comprising circularly permuted IL-2 fused to the extracellular portion of an IL-2Rα chain, wherein the composition comprises a capillary isoelectric focusing (cIEF) profile as depicted in FIG. 15.

In certain embodiments, the composition comprises a cIEF profile peak at one or more of about pI 5.73, about pI 5.93, about pI 6.09, about pI 6.28, about pI 6.38, about pI 6.48, about pI 6.53, about pI 6.66, about pI 6.82, and about pI 7.02.

In certain embodiments, the composition comprises a peak area percent of: about 8% to about 12% at pI 5.93; about 18% to about 26% at pI 6.09; about 22% to about 26% at pI 6.38; and about 18% to about 28% at pI 6.66.

In certain embodiments, the composition comprises a peak area percent of: about 1.5% to about 2.5% at pI 5.73; about 8% to about 12% at pI 5.93; about 18% to about 26% at pI 6.09; about 3.5% to about 4.5% at pI 6.28; about 22% to about 26% at pI 6.38; about 3% to about 5% at pI 6.48; about 4% to about 6% at pI 6.53; about 18% to about 28% at pI 6.66; about 2% to about 6% at pI 6.82; and about 0% to about 3% at pI 7.02.

In certain embodiments, the peak at about pI 5.73 comprises the combination of peaks at about pI 5.70 and about pI 5.76. In certain embodiments, the peak at about pI 5.93 comprises the combination of peaks at about pI 5.89 and about pI 5.97.

In certain embodiments, the composition is produced by the methods recited above.

### BRIEF DESCRIPTION OF THE DRAWINGS

FIG. 1 depicts a process flow diagram for Polypeptide A.

FIG. 2 depicts an SDS-PAGE gel under reducing and non-reducing conditions of samples taken from the bioreactor at specific days.

FIG. 3A-FIG. 3C depict the stability and activity of Polypeptide A after the AEX I purification step. Stability was measured by SDS-PAGE (FIG. 3A) and size exclusion high performance liquid chromatography (SE-HPLC) (FIG. 3B) after the AEX I pool sample was incubated at 5 or 25° C. for 1, 7, or 10 days. The activity was measured in cell-based assay and a pSTAT5 ELISA in dose response curves (FIG. 3C).

FIG. 4 depicts the elevated pH hold step disclosed herein and the Reverse Phase (RP) HPLC traces for samples before and after the elevated pH hold.

FIG. 5 depicts the hydrophobic interaction chromatography (HIC) elution profiles of 4 HIC resins tested herein. The tested resins were Butyl-650 M, Phenyl-650 M, Hexyl-650 C, and PPG-600 M.

FIG. 6A-FIG. 6C depict the stability and activity of Polypeptide A after the HIC purification step. Stability was measured by SDS-PAGE (FIG. 6A) and SE-HPLC (FIG. 6B) after the HIC pool sample was incubated at 5 or 25° C. for 1, 7, or 10 days. The activity was measured in cell-based assay and a pSTAT5 ELISA in dose response curves (FIG. 6C).

FIG. 7A-FIG. 7C depict the stability and activity of Polypeptide A after the MMC purification step. Stability was measured by SDS-PAGE (FIG. 7A) after the HIC pool sample was incubated at 5 or 25° C. for 1, 6, or 11 days. Stability was also measured by SE-HPLC (FIG. 7B) after 4 months at 2-8° C. The activity was measured in cell-based assay and a pSTAT5 ELISA in dose response curves (FIG. 7C).

FIG. 8 depicts an SDS-PAGE gel under reducing and non-reducing conditions of AEX II pool samples held at −80° C., 2-8° C., and ambient temperature.

FIG. 9 depicts two purification schemes in which an elevated pH hold is employed before the AEX I step and after the AEX I step.

FIG. 10 depicts a reverse phase-HPLC (RP-HPLC) trace of the AEX I sample after the harvest material was subjected to the elevated pH hold.

FIG. 11 depicts an RP-HPLC trace of the HIC sample after the harvest material was subjected to the elevated pH hold.

FIG. 12 depicts an RP-HPLC trace of the AEX I sample after the elevated pH hold.

FIG. 13 depicts an RP-HPLC trace of the HIC sample after the elevated pH hold pf the AEX I pool sample.

FIG. 14 depicts the serum concentration (nM) of Polypeptide A in mice over time (hours). The pharmacokinetics of three different lots of purified Polypeptide A were compared. One of the three lots was treated with a sialidase (triangles) while the other two lots were not treated with a sialidase (circles and squares).

FIG. 15 depicts a capillary isoelectric focusing (cIEF) profile of three different lots of purified Polypeptide A.

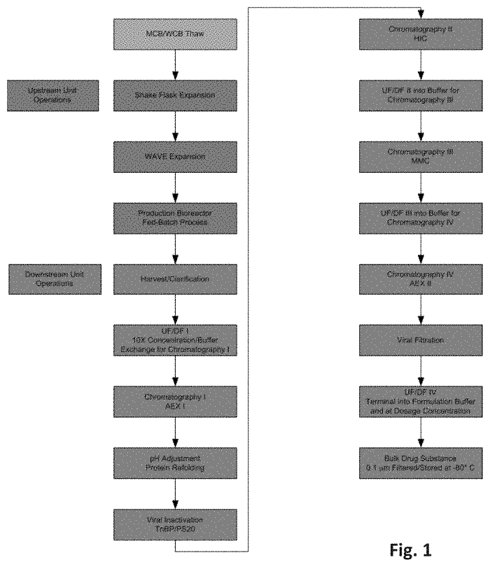

### DETAILED DESCRIPTION

Provided herein are methods of purifying polypeptides comprising a circularly permuted interleukin-2 (IL-2) fused to the extracellular portion of an IL-2Rα chain.

### Selected Definitions

Unless otherwise defined herein, scientific and technical terms used herein have the meanings that are commonly understood by those of ordinary skill in the art. In the event of any latent ambiguity, definitions provided herein take precedent over any dictionary or extrinsic definition. Unless otherwise required by context, singular terms shall include pluralities and plural terms shall include the singular. The use of “or” means “and/or” unless stated otherwise. The use of the term “including,” as well as other forms, such as “includes” and “included,” is not limiting.

As used herein, the terms “circular permutation” and “circularly permuted” refer to the process of taking a linear protein, or its cognate nucleic acid sequence, and fusing the native N- and C-termini (directly or through a linker, using protein or recombinant DNA methodologies) to form a circular molecule, and then cutting (opening) the circular molecule at a different location to form a new linear protein, or cognate nucleic acid molecule, with termini different from the termini in the original molecule. Circular permutation thus preserves the sequence, structure, and function of a protein, while generating new C- and N-termini at different locations that results in an improved orientation for fusing a desired polypeptide fusion partner as compared to the original molecule.

As used herein, the term “about” will be understood by persons of ordinary skill in the art and will vary to some extent on the context in which it is used. As used herein when referring to a measurable value such as an amount, a temporal duration, and the like, the term “about” is meant to encompass variations of ±20% or ±10%, including ±5%, ±1%, and ±0.1% from the specified value, as such variations are appropriate to perform the disclosed methods.

Purification Methods

In one aspect, the instant disclosure provides methods purifying polypeptides comprising a circularly permuted interleukin-2 (IL-2) fused to the extracellular portion of an IL-2Rα chain. Such polypeptides exhibit preferential binding to the intermediate-affinity IL-2R complex comprising IL-2Rβ and the common gamma chain, IL-2Rγ) relative to the high-affinity IL-2R complex (comprising IL-2Rα, IL-2Rβ, and IL-2Rγ), and behave as selective agonists of the intermediate-affinity IL-2R complex. The design and generation of exemplary polypeptides of this type is described in U.S. Pat. No. 9,359,415, which is hereby incorporated by reference in its entirety.

An exemplary polypeptide is set forth below in SEQ ID. NO:1:

(SEQ ID NO: 1)
SKNFHLRPRDLISNINVIVLELKGSETTFMCEYADETATIVEFLNRWITF
SQSIISTLTGGSSSTKKTQLQLEHLLLDLQMILNGINNYKNPKLTRMLTF
KFYMPKKATELKHLQCLEEELKPLEEVLNLAQGSGGGSELCDDDPPEIPH
ATFKAMAYKEGTMLNCECKRGFRRIKSGSLYMLCTGNSSHSSWDNQCQCT
SSATRNTTKQVTPQPEEQKERKTTEMQSPMQPVDQASLPGHCREPPPWEN
EATERIYHFVVGQMVYYQCVQGYRALHRGPAESVCKMTHGKTRWTQPQLI
CTG

Accordingly, in certain embodiments, the amino acid sequence of the polypeptide comprises the amino acid sequence of SEQ ID. NO: 1. In certain embodiments, the amino acid sequence of the polypeptide consists of the amino acid sequence of SEQ ID. NO: 1.

The skilled worker will appreciate that amino acid sequence variants of SEQ ID. NO: 1 can also be employed in the compositions disclosed herein. For example, in certain embodiments, the amino acid sequence of the polypeptide comprises or consists of an amino acid sequence having at least 80% (e.g., 80, 81, 82, 83, 84, 85, 86, 87, 88, 89, 90, 91, 92, 93, 94, 95, 96, 97, 98, or 99%) identity to the amino acid sequence of SEQ ID. NO:1. In certain embodiments, the amino acid sequence of the polypeptide comprises or consists of an amino acid sequence having at least 95% identity to the amino acid sequence of SEQ ID. NO:1.

The skilled worker will also appreciate that amino acid sequence of the polypeptides employed in the compositions disclosed herein can be derivatized or modified, e.g., pegylated, amidated, etc.

In certain embodiments, the purification methods described herein remove or reduce the amount of an impurity. In certain embodiments, the impurity is a host cell protein. The term “host cell protein” (HCP), as used herein, is intended to refer to non-polypeptide proteinaceous impurities derived from host cells, for example, host cells used to produce the polypeptide. In certain embodiments, the HCP may be a HCP derived from a CHO cell. In certain embodiments, the HCP content of a composition of polypeptides comprising a circularly permuted interleukin-2 (IL-2) fused to the extracellular portion of an IL-2Rα chain may be greater than or equal to about 150 ppm, about 200 ppm, about 250 ppm, about 300 ppm, about 350 ppm, about 400 ppm, about 450 ppm, about 500 ppm, about 1000 ppm, about 1500 ppm, about 2000 ppm, about 2500 ppm, or about 3000 ppm. HCP content may alternatively be described by units of ng/mL. In certain embodiments, a “partially purified” polypeptide is a polypeptide that is part of a composition comprising greater than or equal to about 150 ppm HCP content that has been previously subjected to one or more purification techniques. The partially purified polypeptide may be subjected to further purification techniques to further reduce the HCP content. In certain embodiments, the HCP content is reduced to less than or equal to about 100 ppm, about 75 ppm, about 50 ppm, or about 25 ppm.

In certain embodiments, the impurity is a host cell nucleic acid. The term “host cell nucleic acids”, as used herein, is intended to refer to nucleic acids derived from host cells, for example, host cells used to produce a polypeptide disclosed herein.

In a particular embodiment, the impurity is a product-related substance. As used herein, the term “product-related substance” refers to a variant of a polypeptide disclosed herein that is formed during the manufacturing and/or storage of the polypeptide. Specific examples of product-related substances include degradants of the polypeptide, truncated forms of the polypeptide, high molecular weight species, low molecular weight species, fragments of the polypeptide, modified forms of the polypeptide, including deamidated, isomerized, mismatched disuphide linked, oxidized or altered conjugate forms (e.g., glycosylation, phosphorylation), aggregates including dimers and higher multiples of the polypeptide, and charge variants. In a particular embodiment, the impurity is an aggregate of the polypeptide. As used herein, the term “aggregate” refers to agglomeration or oligomerization of two or more individual molecules of the polypeptide to form, for example, dimers, trimers, tetramers, oligomers and other high molecular weight species. Aggregates can be soluble or insoluble. In a particular embodiment, the aggregate is a multimer of a polypeptide disclosed herein. In a particular embodiment, the aggregate is a multimer of a polypeptide comprising the amino acid sequence of SEQ ID NO: 1.

In certain embodiments, the composition of polypeptides comprising a circularly permuted interleukin-2 (IL-2) fused to the extracellular portion of an IL-2Rα chain may be purified with a purity of greater than or equal to about 80%, about 85%, about 90%, about 95%, about 96%, about 97%, about 98%, about 99%, or about 100%. In certain embodiments, the purity is determined by Reverse Phase (RP) HPLC.

### Ion Exchange Chromatography (IEX)

In certain embodiments, a sample comprising a polypeptide disclosed herein is subjected to purification by ion exchange chromatography (IEX). Additionally or alternatively, the wash and/or flow through fractions generated by the methods of the present disclosure can be subjected to ion exchange chromatography to further purify the polypeptide. In certain embodiments, one or more ion exchange chromatography steps are performed to purify the polypeptide. In certain embodiments, one or more ion exchange chromatography steps are performed prior to a hydrophobic interaction chromatography (HIC) purification step. In certain embodiments, one or more ion exchange chromatography steps are performed after a HIC purification step.

As used herein, ion exchange separation includes any method by which two substances are separated based on the difference in their respective ionic charges, either on the polypeptide and/or chromatographic material as a whole or locally on specific regions of the polypeptide and/or chromatographic material, and thus can employ either cationic exchange (CEX) material or anionic exchange (AEX) material.

The use of a cationic exchange material versus an anionic exchange material is based on the local charges of the polypeptide in a given solution. Therefore, it is within the scope of this disclosure to employ an anionic exchange step or a cationic exchange step. Furthermore, it is within the scope of this disclosure to employ only a cationic exchange step, only an anionic exchange step, or any serial combination of the two either prior to or subsequent to any other chromatography step, such as a HIC step or MMC step.

In performing the separation, the sample containing a polypeptide disclosed herein (e.g., a circularly permuted IL-2 fused to the extracellular portion of an IL-2Rα chain) can be contacted with the ion exchange material by using any of a variety of techniques, e.g., using a batch purification technique or a chromatographic technique. Furthermore, the sample containing the polypeptide can be contacted with the ion exchange material in a bind-elute mode, wherein the polypeptide binds to the ion exchange resin, allowing impurities to flow through the resin or bind the resin more weakly than the polypeptide. A wash step may be performed to remove or reduce the impurities weakly bound to the ion exchange resin. A subsequent elution step may be performed to remove the polypeptide from the ion exchange resin as part of an eluate, or pool. The eluate or pool may be made up of multiple fractions or from one large elution. Alternatively, the sample containing the polypeptide can be contacted with the ion exchange material in a flow-through mode, wherein the polypeptide does not bind or binds weakly to the ion exchange resin, allowing impurities to bind the resin or bind the resin stronger than the polypeptide. A wash or elution step may not be performed in a flow-through mode.

Ion exchange chromatography separates molecules based on differences between the local charges of the polypeptides of interest and the local charges of the chromatographic material. A packed ion-exchange chromatography column or an ion-exchange membrane device can be operated in a bind-elute mode, a flow-through, or a hybrid mode. After washing the column or the membrane device with the equilibration buffer or another buffer with different pH and/or conductivity, the product recovery is achieved by increasing the ionic strength (i.e., conductivity) of the elution buffer to compete with the solute for the charged sites of the ion exchange matrix. Changing the pH and thereby altering the charge of the solute is another way to achieve elution of the solute. The change in conductivity or pH may be gradual (gradient elution) or stepwise (step elution). The column is then regenerated before next use.

In certain embodiments, the gradient elution conditions comprise one or more of: increasing the conductivity of an elution buffer over time; increasing the salt concentration of an elution buffer over time; or decreasing the pH of an elution buffer over time. In certain embodiments, the gradient elution buffer comprises an initial conductivity of less than or equal to about 5 mS/cm. In certain embodiments, the gradient elution buffer comprises an initial conductivity of about 5 mS/cm, about 4 mS/cm, about 3 mS/cm, about 2 mS/cm, about 1 mS/cm, or about 0 mS/cm. In certain embodiments, the gradient elution buffer comprises a final conductivity of greater than or equal to about 15 mS/cm. In certain embodiments, the gradient elution buffer comprises a final conductivity of about 15 mS/cm, about 16 mS/cm, about 17 mS/cm, about 18 mS/cm, about 19 mS/cm, about 20 mS/cm, about 30 mS/cm, about 40 mS/cm, about 50 mS/cm, about 60 mS/cm, about 70 mS/cm, about 80 mS/cm, about 90 mS/cm, or about 100 mS/cm. In certain embodiments, the conductivity of the elution buffer is increased from about ≤2 mS/cm to about ≥15 mS/cm. In certain embodiments, the conductivity of the elution buffer is increased from about ≤2 mS/cm to about ≥20 mS/cm. In certain embodiments, the conductivity of the elution buffer is increased to between about 15 mS/cm to about 100 mS/cm. In certain embodiments, the conductivity of the elution buffer is increased to between about 20 mS/cm to about 50 mS/cm.

In certain embodiments, the gradient elution buffer comprises an initial salt concentration of about 0 M salt, about 0.01 M salt, about 0.02 M salt, about 0.03 M salt, about 0.04 M salt, or about 0.05 M salt. In certain embodiments, the gradient elution buffer comprises a final salt concentration of about 0.15 M salt, about 0.20 M salt, about 0.25 M salt, about 0.30 M salt, about 0.35 M salt, about 0.40 M salt, about 0.45 M salt, or about 0.50 M salt. In certain embodiments, the salt concentration of the elution buffer is increased from about 0 M salt to about 1.0 M salt. In certain embodiments, the salt concentration of the elution buffer is increased from about 0 M salt to about 0.5 M salt.

In certain embodiments, the salt comprises sodium chloride, potassium chloride, magnesium chloride, or calcium chloride. In certain embodiments, the salt comprises sodium chloride.

In certain embodiments, the elution buffer further comprises a pH of about 7.0 to about 9.0. In certain embodiments, the elution buffer further comprises a pH of about 8.0.

Anionic or cationic substituents may be attached to matrices in order to form anionic or cationic supports for chromatography. Non-limiting examples of anionic exchange substituents include diethylaminoethyl (DEAE), quaternary aminoethyl (QAE) and quaternary amine (Q) groups. Cationic substituents include carboxymethyl (CM), sulfoethyl (SE), sulfopropyl (SP), phosphate (P) and sulfonate (S). Cellulose ion exchange medias such as DE23, DE32, DE52, CM-23, CM-32, and CM-52 are available from Whatman Ltd. Maidstone, Kent, U.K. SEPHADEX-based and -locross-linked ion exchangers are also known. For example, DEAE-, QAE-, CM-, and SP-SEPHADEX and DEAE-, Q-, CM- and S-SEPHAROSE and SEPHAROSE, Fast Flow, and Capto S are all available from GE Healthcare. Further, both DEAE and CM derivatized ethylene glycol-methacrylate copolymer such as TOYOPEARL, DEAE-650S or M and TOYOPEARL CM-650S or M are available from Toso Haas Co., Philadelphia, Pa., or Nuvia S and UNOSphere S from BioRad, Hercules, Calif., Eshmuno S from EMD Millipore, Billerica, Calif. Further, the TOYOPEARL GigaCap Q-650M is available from Tosoh Biosciences.

### High pH Refolding

In certain embodiments, the methods described herein comprise an elevated pH hold step, e.g., after an initial IEX (e.g., AEX or CEX) purification step and prior to an additional purification step (e.g., a HIC purification step). Without being bound to any theory, it is believed that, in certain embodiments, this elevated pH hold step promotes refolding of the polypeptide and reduces the level of a product-related substance impurity. In certain embodiments, the elevated pH hold step is performed by adjusting the pH of a polypeptide-containing sample to about 10.0 to about 12.0. In certain embodiments, the pH is adjusted to about 10.0, about 10.1, about 10.2, about 10.3, about 10.4, about 10.5, about 10.6, about 10.7, about 10.8, about 10.9, about 11.0, about 11.1, about 11.2, about 11.3, about 11.4, about 11.5, about 11.6, about 11.7, about 11.8, about 11.9, or about 12.0. In certain embodiments the pH is adjusted to ≥10.7. In certain embodiments, the pH is adjusted to about 10.8.

In certain embodiments, the pH is adjusted by adding a base or a basic buffer such as sodium carbonate and/or sodium hydroxide. The skilled worker will readily understand the appropriate manner in which to adjust the pH.

In certain embodiments, a sample comprising a polypeptide disclosed herein is held at an elevated pH (e.g., a pH of about 10.8) for an incubation period. The incubation period may be, for example, about 30 minutes to about 3 hours. The incubation period may be about 30 minutes, 45 minutes, 60 minutes, 75 minutes, 90 minutes, 105 minutes, 120 minutes, 135 minutes, 150 minutes, 165 minutes, or 180 minutes. In a certain embodiment, the incubation time may be about or at least 60 minutes. In certain embodiments, the sample comprising a polypeptide disclosed herein is held at an elevated pH (e.g., a pH of about 10.8) for an incubation period while mixing the sample.

After the sample has been incubated for an appropriate period at the elevated pH, the pH of the sample may be lowered by the addition of an acid, including but not limited to, citric acid. In certain embodiments, the pH of the sample is lowered to a pH of about 8.0 to about 9.0. In certain embodiments, the pH of the sample is lowered to a pH of about 8.0, about 8.1, about 8.2, about 8.3, about 8.4, about 8.5, about 8.6, about 8.7, about 8.8, about 8.9, or about 9.0. In certain embodiments, the pH of the sample is lowered to about 8.5. The skilled worker will readily understand the appropriate manner in which to adjust the pH of a sample to lower the pH.

### Hydrophobic Interaction Chromatography (HIC)

In certain embodiments, a sample comprising a polypeptide disclosed herein is subjected to hydrophobic interaction chromatography (HIC) to purify the polypeptide. Additionally or alternatively, the wash and/or flow through fractions generated by the methods of the present disclosure can be subjected to HIC to further purify the polypeptide. In certain embodiments, one or more HIC steps are performed to purify the polypeptide. In certain embodiments, one or more ion exchange chromatography steps are performed prior to a HIC purification step. In certain embodiments, one or more ion exchange chromatography steps are performed after a HIC purification step.

HIC purification of a polypeptide comprises reversible binding of the polypeptide and binding of one or more impurities through hydrophobic interaction with hydrophobic moieties attached to a solid matrix support (e.g., agarose). The hydrophobic interaction between molecules results from the tendency of a polar environment to exclude non-polar (i.e., hydrophobic) molecules. HIC relies on this principle of hydrophobicity of molecules (i.e., the tendency of a given protein to bind adsorptively to hydrophobic sites on a hydrophobic adsorbent body) to separate biomolecules based on their relative strength of interaction with the hydrophobic moieties (see, e.g., U.S. Pat. Nos. 4,000,098 and 3,917,527). An advantage of this separation technique is its non-denaturing characteristics and the stabilizing effects of salt solutions used during loading, washing and or eluting.

Hydrophobic interaction chromatography employs the hydrophobic properties of molecules (e.g., proteins, polypeptides, lipids) to achieve separation of even closely-related molecules. Hydrophobic groups on the molecules interact with hydrophobic groups of the media or the membrane. In certain embodiments, the more hydrophobic a molecule is, the stronger it will interact with the column or the membrane. Thus, HIC steps, such as those disclosed herein, can be used to remove a variety of impurities, for example, process-related impurities (e.g., DNA) as well as product-related species (e.g., high and low molecular weight product-related species, such as protein aggregates and fragments).

In certain embodiments, the HIC adsorbent material is composed of a chromatographic backbone with pendant hydrophobic interaction ligands. For example, but not by way of limitation, the HIC media can be composed of convective membrane media with pendent hydrophobic interaction ligands, convective monolithic media with pendent hydrophobic interaction ligands, and/or convective filter media with embedded media containing the pendant hydrophobic interaction ligands.

In certain embodiments, the HIC adsorbent material can comprise a base matrix (e.g., derivatives of cellulose, polystyrene, synthetic poly amino acids, synthetic polyacrylamide gels, cross-linked dextran, cross-linked agarose, synthetic copolymer material or even a glass surface) to which hydrophobic ligands (e.g., alkyl, aryl and combinations thereof) are coupled or covalently attached using difunctional linking groups such as —NH—, —S—, —COO—, etc. The hydrophobic ligand may be terminated in a hydrogen but can also terminate in a functional group such as, for example, NH₂, SO₃H, PO₄H₂, SH, imidazoles, phenolic groups or non-ionic radicals such as OH and CONH₂. In certain embodiments, the HIC media comprises at least one hydrophobic ligand. In another embodiment, the hydrophobic ligand is selected from the group consisting of butyl, hexyl, phenyl, octyl, or polypropylene glycol ligands.

One, non-limiting, example of a suitable HIC media comprises an agarose media or a membrane functionalized with phenyl groups (e.g., a Phenyl Sepharose from GE Healthcare or a Phenyl Membrane from Sartorius). Many HIC medias are available commercially. Examples include, but are not limited to, Tosoh Hexyl, CaptoPhenyl, Phenyl Sepharose 6 Fast Flow with low or high substitution, Phenyl Sepharose High Performance, Octyl Sepharose High Performance (GE Healthcare); Fractogel EMD Propyl or Fractogel EMD Phenyl (E. Merck, Germany); Macro-Prep Methyl or Macro-Prep t-Butyl columns (Bio-Rad, California); WP HI-Propyl (C3) (J. T. Baker, New Jersey); Toyopearl ether, phenyl or butyl (TosoHaas, PA); ToyoScreen PPG, Toyopearl PPG-600M, ToyoScreen Phenyl, ToyoScreen Butyl, and ToyoScreen Hexyl are a rigid methacrylic polymer bead. GE HiScreen Butyl FF and HiScreen Octyl FF are high flow agarose based beads.

### Mixed Mode Chromatography (MMC)

In certain embodiments, a sample comprising a polypeptide disclosed herein is subjected to mixed mode chromatography (MMC) to purify the polypeptide. Additionally or alternatively, the wash and/or flow through fractions generated by the methods of the present invention can be subjected to mixed mode chromatography to further purify the polypeptide. In certain embodiments, one or more mixed mode chromatography steps may be used after a HIC purification step. In certain embodiments, one or more mixed mode chromatography steps may be used prior to a HIC purification step. In certain embodiments, one or more mixed mode chromatography steps may be used after an ion exchange purification step. In certain embodiments, one or more mixed mode chromatography steps may be used prior to an ion exchange purification step.

Mixed mode chromatography is chromatography that utilizes a mixed mode media, including, but not limited to, CaptoAdhere or Capto MMC ImpRes available from GE Healthcare. Such a media comprises a mixed mode chromatography ligand. In certain embodiments, such a ligand refers to a ligand that is capable of providing at least two different, but co-operative, sites which interact with the substance to be bound. One of these sites gives an attractive type of charge-charge interaction between the ligand and the polypeptide. The other site typically gives electron acceptor-donor interaction and/or hydrophobic and/or hydrophilic interactions. Electron donor-acceptor interactions include interactions such as hydrogen-bonding, π-π, cation-π, charge transfer, dipole-dipole, induced dipole etc. The mixed mode functionality can give a different selectivity compared to traditional anion exchangers. Mixed mode chromatography ligands are also known as “multimodal” chromatography ligands. Capto Adhere comprises a rigid, high-flow agarose matrix functionalized with N-benzyl-N-methyl ethanolamine ligand. Capto MMC comprises a rigid, high-flow agarose matrix functionalized with benzoylhomocysteine ligand.

In certain embodiments, the mixed mode chromatography media is comprised of mixed mode ligands coupled to an organic or inorganic support, sometimes denoted a base matrix, directly or via a spacer. In certain embodiments, the mixed mode ligand may be a multimodal weak cation exchanger. The support may be in the form of particles, such as essentially spherical particles, a monolith, filter, membrane, surface, capillaries, etc. In certain embodiments, the support is prepared from a native polymer, such as cross-linked carbohydrate material, such as agarose, agar, cellulose, dextran, chitosan, konjac, carrageenan, gellan, alginate etc. To obtain high adsorption capacities, the support can be porous, and ligands are then coupled to the external surfaces as well as to the pore surfaces. Such native polymer supports can be prepared according to standard methods, such as inverse suspension gelation (S Hjerten: Biochim Biophys Acta 79(2), 393-398 (1964). Alternatively, the support can be prepared from a synthetic polymer, such as cross-linked synthetic polymers, e.g. styrene or styrene derivatives, divinylbenzene, acrylamides, acrylate esters, methacrylate esters, vinyl esters, vinyl amides etc. Such synthetic polymers can be produced according to standard methods, see e.g. “Styrene based polymer supports developed by suspension polymerization” (R Arshady: Chimica e L'Industria 70(9), 70-75 (1988)). Porous native or synthetic polymer supports are also available from commercial sources, such as Amersham Biosciences, Uppsala, Sweden.

### Viral Filtration

In certain embodiments, a sample comprising a polypeptide disclosed herein is subjected to viral filtration to further purify the polypeptide. Additionally or alternatively, the wash and/or flow through fractions generated by the methods of the present invention can be subjected to viral filtration to further purify the polypeptide.

Viral filtration is a dedicated viral reduction step in the entire purification process. In certain embodiments, this step is performed as a post chromatographic polishing step. Viral reduction can be achieved via the use of suitable filters including, without limitation, Planova 20N, 50 N or BioEx from Asahi Kasei Pharma, Viresolve filters from EMD Millipore, ViroSart CPV from Sartorius, or Ultipor DV20 or DV50 filter from Pall Corporation. It will be apparent to one of ordinary skill in the art to select a suitable filter to obtain desired filtration performance.

### Ultrafiltration/Diafiltration

In certain embodiments, the methods of purifying a polypeptide described herein may comprises one or more ultrafiltration (UF) and/or diafiltration (DF) steps to concentrate the polypeptide and exchange the buffer of the polypeptide. In certain embodiments, the ultrafiltration step may concentrate the polypeptide by a factor of about 2× to about 100×. In certain embodiments, the ultrafiltration step may concentrate the polypeptide by a factor of about 2×, about 5×, about 10×, about 20×, about 30×, about 40×, about 50×, about 60×, about 70×, about 80×, about 90×, or about 100×. In certain embodiments, the ultrafiltration step may concentrate the polypeptide by a factor of about 10×.

Ultrafiltration is described in detail in, e.g., Microfiltration and Ultrafiltration: Principles and Applications, L. Zeman and A. Zydney (Marcel Dekker, Inc., New York, N.Y., 1996); and in: Ultrafiltration Handbook, Munir Cheryan (Technomic Publishing, 1986; ISBN No. 87762-456-9). One filtration process is Tangential Flow Filtration, e.g., as described in the Millipore catalogue entitled “Pharmaceutical Process Filtration Catalogue” pp. 177-202 (Bedford, Mass., 1995/96). Ultrafilters include, without limitation, the Sartorius Hydrosart ultrafilters. In certain embodiments, ultrafiltration includes filtration using filters with a pore size of smaller than 0.1 μm. By employing filters having such small pore size, the volume of the sample can be reduced through permeation of the sample buffer through the filter while polypeptides of interest are retained behind the filter. Ultrafilters may be defined by Molecular Weight Cut Off (MWCO) values, such as a MWCO of 2 kD, 5 kD, 10 kD, 30 kD, or 100 kD. In certain embodiments, the ultrafilters may have a MWCO of 10 kD.

Diafiltration is a method of using ultrafilters to remove and exchange salts, sugars, and non-aqueous solvents, to separate free from bound species, to remove low molecular-weight material, and/or to cause the rapid change of ionic and/or pH environments. Microsolutes are removed most efficiently by adding solvent to the solution being ultrafiltered at a rate approximately equal to the ultratfiltration rate. This washes microspecies from the solution at a constant volume, effectively purifying the retained polypeptides of interest. In certain embodiments of the methods disclosed herein, a diafiltration step is employed to exchange the various buffers employed, optionally prior to further chromatography or other purification steps, as well as to remove impurities from the polypeptide preparations.

### Complementary Purification Techniques

In certain embodiments, a combination of ion exchange chromatography, mixed mode chromatography, and hydrophobic interaction chromatography methods may be used to prepare preparations of the polypeptide having a reduced level of impurity, including certain embodiments where one technology is used in a complementary/supplementary manner with another technology. In certain embodiments, such combinations include the use of additional intervening chromatography, filtration, pH adjustment, UF/DF (ultrafiltration/diafiltration) steps to achieve the desired product quality, ion concentration, and/or viral reduction.

In certain embodiments, a polypeptide disclosed herein may be purified from a host cell culture by following a particular purification method or scheme. In certain embodiments, the polypeptide (e.g., a circularly permuted IL-2 fused to the extracellular portion of an IL-2Rα chain) may be purified through the following sequential chromatography steps: 1) a first anion exchange chromatography (AEX) step, 2) a hydrophobic interaction chromatography (HIC) step, 3) a mixed mode chromatography (MMC) step, and 4) a second AEX step. One or more filtration steps may be employed at any point in the chromatography steps described above. In certain embodiments, the polypeptide may be purified through the following sequential purification steps: 1) a first ultrafiltration/diafiltration step, 2) a first anion exchange chromatography (AEX) step, 3) a high pH incubation step (e.g., incubation at pH of about 10.8 for at least 60 min), 4) a viral inactivation step, 5) a hydrophobic interaction chromatography (HIC) step, 6) a second ultrafiltration/diafiltration step, 7) a mixed mode chromatography (MMC) step, 8) a third ultrafiltration/diafiltration step, 9) a second AEX step, 10) a viral filtration step, and 11) a fourth ultrafiltration/diafiltration step.

Polypeptide Compositions with Specific Glycan Profiles and Methods of Improving Serum Half-Life

In certain embodiments, the purification methods described herein may be used to improve the serum half-life of the polypeptide composition. The purified composition comprises a plurality of polypeptides, each polypeptide of the plurality comprising the amino sequence of SEQ ID NO: 1 linked to one or more glycan species, wherein the one or more glycan species are linked to the polypeptide at one or more of amino acid positions N187, N206, and T212 of SEQ ID NO: 1. The amount and type of glycan species on the polypeptides of the composition may impact the serum half-life of the composition after being administered to a patient. The glycan profile of the composition is the percent of each glycan species at amino acid positions N187, N206, and T212 of SEQ ID NO: 1 among all of the polypeptides combined. Alterations to the composition glycan profile may increase or decrease the serum half-life of the composition.

In certain embodiments, the glycan species at amino acid position N187 of SEQ ID NO: 1 are selected from the group consisting of:
Hex5HexNAc4FucNeuAc2;
Hex6HexNAc5FucNeuAc2;
Hex5HexNAc4FucNeuAc;
Hex6HexNAc5FucNeuAc3;
Hex4HexNAc4FucNeuAc;
Hex5HexNAc5NeuAc2;
Hex5HexNAc4Fuc;
Hex3HexNAc4Fuc;
Hex4HexNAc4Fuc;
Hex6HexNAc5Fuc; and
Hex5HexNAc5Fuc;
   wherein Hex represents hexose, HexNAc represents N-acetylhexosamine, NeuAc represents N-acetylneuraminic acid, Fuc represents fucose, and the number represents the number of each glycan structure.

In certain embodiments, the glycan species at amino acid position N206 of SEQ ID NO: 1 are selected from the group consisting of:
Hex6HexNAc5FucNeuAc3;
Hex5HexNAc4FucNeuAc2;
Hex6HexNAc5FucNeuAc2;
Hex7HexNAc6FucNeuAc3;
Hex6HexNAc5FucNeuAc;
Hex5HexNAc4FucNeuAc;
Hex5HexNAc4Fuc; and
Hex4HexNAc4Fuc;
   wherein Hex represents hexose, HexNAc represents N-acetylhexosamine, NeuAc represents N-acetylneuraminic acid, Fuc represents fucose, and the number represents the number of each glycan structure.

In certain embodiments, the glycan species at amino acid position T212 of SEQ ID NO: 1 are selected from the group consisting of:
HexHexNAc;
HexHexNAcNeuAc; and
HexHexNAcNeuAc2;
   wherein Hex represents hexose, HexNAc represents N-acetylhexosamine, NeuAc represents N-acetylneuraminic acid, and the number represents the number of each glycan structure.

In certain embodiments, the overall percent of glycan species at amino acid position N187 of SEQ ID NO: 1 of the plurality of polypeptides in the composition comprises:
about 60% to about 70% Hex5HexNAc4FucNeuAc2, such as about 60%, about 61%; about 62%, about 63%, about 64%, about 65%, about 66%, about 67%, about 68%, about 69%, or about 70%;
about 4% to about 6% Hex6HexNAc5FucNeuAc2, such as about 4%, about 5%, or about 6%;
about 7% to about 10% Hex5HexNAc4FucNeuAc, such as about 7%, about 8%, about 9%, or about 10%;
about 15% to about 17% Hex6HexNAc5FucNeuAc3, such as about 15%, about 16%, or about 17%; and
about 3% to about 4% Hex5HexNAc5NeuAc2.

In certain embodiments, the overall percent of glycan species at amino acid position N187 of SEQ ID NO: 1 of the plurality of polypeptides in the composition comprises:
about 60% to about 70% Hex5HexNAc4FucNeuAc2;
about 4% to about 6% Hex6HexNAc5FucNeuAc2;
about 7% to about 10% Hex5HexNAc4FucNeuAc;
about 15% to about 17% Hex6HexNAc5FucNeuAc3;
about 0.5% to about 1.5% Hex4HexNAc4FucNeuAc;
about 3% to about 4% Hex5HexNAc5NeuAc2;
about 0% to about 0.5% Hex5HexNAc4Fuc;
about 0% to about 0.5% Hex3HexNAc4Fuc;
about 0% to about 0.5% Hex4HexNAc4Fuc;
about 0% to about 0.5% Hex6HexNAc5Fuc; and
about 0% to about 0.5% Hex5HexNAc5Fuc.

In certain embodiments, the overall percent of glycan species at amino acid position N206 of SEQ ID NO: 1 of the plurality of polypeptides in the composition comprises:
about 3% to about 5% Hex6HexNAc5FucNeuAc3, such as about 3%, about 4%, or about 5%;
about 75% to about 85% Hex5HexNAc4FucNeuAc2, such as about 75%, about 76%; about 77%, about 78%, about 79%, about 80%, about 81%, about 82%, about 83%, about 84%, or about 85%;
about 2% to about 4% Hex6HexNAc5FucNeuAc2, such as about 2%, about 3%, or about 4%;
about 5% to about 12% Hex5HexNAc4FucNeuAc, such as about 5%, about 6%, about 7%, about 8%, about 9%, about 10%, about 11%, or about 12%; and
about 1% to about 3% Hex5HexNAc4Fuc, such as about 1%, about 2%, or about 3%.

In certain embodiments, the overall percent of glycan species at amino acid position N206 of SEQ ID NO: 1 of the plurality of polypeptides in the composition comprises:
about 3% to about 5% Hex6HexNAc5FucNeuAc3;
about 75% to about 85% Hex5HexNAc4FucNeuAc2;
about 2% to about 4% Hex6HexNAc5FucNeuAc2;
about 0.5% to about 1.5% Hex7HexNAc6FucNeuAc3;
about 0% to about 1% Hex6HexNAc5FucNeuAc;
about 5% to about 12% Hex5HexNAc4FucNeuAc;
about 1% to about 3% Hex5HexNAc4Fuc; and
about 0.5% to about 2% Hex4HexNAc4Fuc.

In certain embodiments, the overall percent of glycan species at amino acid position T212 of SEQ ID NO: 1 of the plurality of polypeptides in the composition comprises:
about 14% to about 18% HexHexNAcNeuAc, such as about 14%, about 15%, about 16%, about 17%, or about 18%; and
about 8% to about 13% HexHexNAcNeuAc2, such as about 8%, about 9%, about 10%, about 11%, about 12%, or about 13%.

In certain embodiments, the overall percent of glycan species at amino acid position T212 of SEQ ID NO: 1 of the plurality of polypeptides in the composition comprises:
about 0% to about 1% HexHexNAc;
about 14% to about 18% HexHexNAcNeuAc; and
about 8% to about 13% HexHexNAcNeuAc2.

In certain embodiments, the overall percent of an unglycosylated amino acid at amino acid position N206 of SEQ ID NO: 1 of the plurality of polypeptides in the composition comprises about 65% to about 80%.

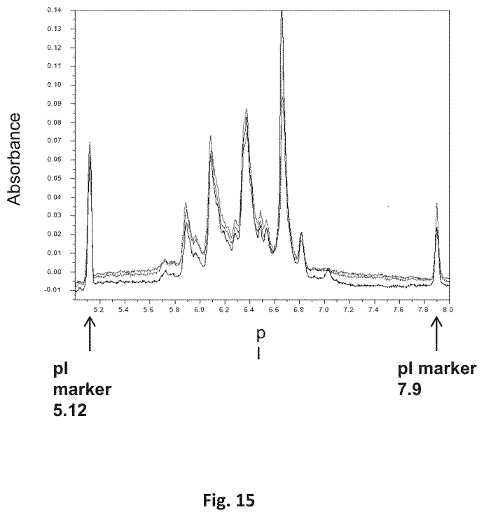

In certain embodiments, the purification methods described herein may be used to provide a composition comprising a plurality of polypeptides, each polypeptide of the plurality comprising circularly permuted IL-2 fused to the extracellular portion of an IL-2Rα chain, wherein the composition comprises a specific capillary isoelectric focusing (cIEF) profile, e.g., the cIEF profile as depicted in FIG. 15. The cIEF profile is a fingerprint of the charge heterogeneity of the composition of a plurality of polypeptides.

In certain embodiments, the composition comprises a cIEF profile peak at one or more of about pI 5.73, about pI 5.93, about pI 6.09, about pI 6.28, about pI 6.38, about pI 6.48, about pI 6.53, about pI 6.66, about pI 6.82, and about pI 7.02.

In certain embodiments, the composition comprises a peak area percent of:
about 8% to about 12% at pI 5.93;
about 18% to about 26% at pI 6.09;
about 22% to about 26% at pI 6.38; and
about 18% to about 28% at pI 6.66.

In certain embodiments, the composition comprises a peak area percent of:
about 1.5% to about 2.5% at pI 5.73;
about 8% to about 12% at pI 5.93;
about 18% to about 26% at pI 6.09;
about 3.5% to about 4.5% at pI 6.28;
about 22% to about 26% at pI 6.38;
about 3% to about 5% at pI 6.48;
about 4% to about 6% at pI 6.53;
about 18% to about 28% at pI 6.66;
about 2% to about 6% at pI 6.82; and
about 0% to about 3% at pI 7.02.

In certain embodiments, the peak at about pI 5.73 comprises the combination of peaks at about pI 5.70 and about pI 5.76. In certain embodiments, the peak at about pI 5.93 comprises the combination of peaks at about pI 5.89 and about pI 5.97.

It will be readily apparent to those skilled in the art that other suitable modifications and adaptations of the methods described herein may be made using suitable equivalents without departing from the scope of the embodiments disclosed herein. Having now described certain embodiments in detail, the same will be more clearly understood by reference to the following examples, which are included for purposes of illustration only and are not intended to be limiting.

### EXAMPLES

The invention is further illustrated by the following examples, which should not be construed as further limiting. The practice of the present invention will employ, unless otherwise indicated, conventional techniques of organic synthesis, cell biology, cell culture, molecular biology, transgenic biology, microbiology and immunology, which are within the skill of the art.

### Example 1—Upstream Polypeptide A Production

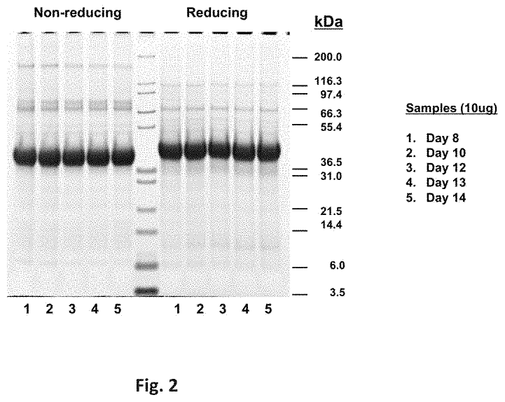

Polypeptide A was produced from a single cell clone of the CHOK1SV cell line transfected to express Polypeptide A. The upstream seed train process for Polypeptide A production went from a cryogenically stored cell bank (about 1×10⁷ viable cells) to a minimal critical mass of about 5×10¹⁰ viable cells at >95% viability to inoculate a 200 L bioreactor at about 3×10⁵ viable cells/mL (minimum seed density) to 70 percent working volume (140 L) production media. To reach this end critical mass, the cells were expanded through incrementally increasing sized sterile shake flasks and rocker bags in 3 to 4 day intervals. The scale up and Polypeptide A production took place over about 10-14 days before downstream cell culture harvesting and purification was conducted. SDS-PAGE was performed at day 8, 10, 12, 13, and 14 to determine relative purity and abundance of Polypeptide A (FIG. 2).

### Example 2—Downstream Polypeptide A Production/Purification

### Harvest and Clarification

Cell culture harvest and clarification were performed using EMD POD depth filters followed by a final clarification through a Durapore 10″ 0.2 μm cartridge filter.

### Ultrafiltration (UF)/Diafiltration (DF) I

The UF and DF processing steps were used for product concentration and buffer exchange, respectively. During manufacturing, to prevent any potential carry over between UF/DF unit operations, all UF/DF cassettes were used only once. Based on the size of Polypeptide A (˜40 kD) a Sartorious Hydrosart 10 kD molecular weight cut off (MWCO) membrane cassette was used in all three UF/DF steps. The first two UF/DF steps were primarily used to concentrate the dilute product solution and to perform a buffer exchange making them suitable for further processing by column chromatography steps, while the final UF/DF step was used to put the purified protein into formulation and buffer and bring it to final concentration prior to drug product manufacturing.

The first UF step was used to concentration the product by a factor or about 10× or more. The cassettes were flushed with WFI and PBS to remove the storage solution. The clarified harvest was supplied at a flow rate of 600-700 L/H; feed inlet pressure is maintained between 15-29 psi, retentate pressure is between 10-14 psi, and the trans membrane pressure (TMP) was maintained between 12.5-21.5 psi. After concentration, the process stream was diafiltered into 20 mM Tris, pH 8.5±0.5 buffer with a 1-2 mS conductivity for 5 DV. The final recovered product solution was tested via SE-HPLC and RP-HPLC for purity and concentration respectively. Overall yield for this step was typically >90% and the product solution was stored between 2-8° C. until further processing.

### Anion Exchange Chromatography (AEX) I

Following the UF/DF I step, a first AEX step (AEX I) was employed to capture Polypeptide A from the clarified harvest. The GigaBap Q 650 M resin (Tosoh Biosciences) was used with a 20 cm packed bed and a Dynamic Binding Capacity (DBC) of 30 g/L resin. The AEX column was first equilibrated with alternating single column volumes of AEX I buffer B (AEX B) and AEX I buffer A (AEX A) for 4 total column volumes, at a flow rate of 300 cm/hr.

The UF/DF I step product was loaded onto the AEX I column at a flow rate of 300 cm/hr. Following the loading step, the column was washed with AEX I buffer C (AEX C) for 5 column volumes and a flow rate of 300 cm/hr.

Polypeptide A was then eluted from the AEX I column with AEX I buffer D (AEX D) with 10 column volumes and a flow rate of 300 cm/hr. Elutions were collected with an elution peak cutoff of 300 mAU. Table 1 below recites the AEX I buffers used. Table 2 below shows a comparison of the AEX I load to the AEX I pooled elution sample. Polypeptide A amount, step yield, and CHO Host Cell Protein (HCP) amount are shown.

TABLE 1
AEX I buffers

| Buffer | Buffer | | Conductivity | |
|---|---|---|---|---|
| name | composition | pH | (mS/cm.) | Function |
| AEX A | 20 mM Tris- | 8.5 +/− 0.1 | 1-2 | Column EQ, |
| | HCl | | | Load |
| AEX B | 20 mM Tris- | 8.5 +/− 0.1 | 80-92 | Column EQ, |
| | HC1 + 1M | | | Strip |
| | NaCl | | | |
| AEX C | 20 mM Tris- | 7.5 +/− 0.1 | 1-2 | Column Wash |
| | HCl | | | |
| AEX D | 20 mM Tris- | 8.5 +/− 0.1 | ~20 (18-24) | Column |
| | HCl + 0.2M | | | Elution |
| | NaCl | | | |

TABLE 2
AEX I purification step results

| | | | Poly- | Step | | |
|---|---|---|---|---|---|---|
| | Titer | Volume | peptide | yield | CHO HCP | |
| Description | (mg/mL) | (L) | A (g) | (%) | ng/mL | ppm |
| AEX I Load | 11.36 | 15.6 | 177 | N/A | 789380 | 69488 |
| AEX I Pool | 15.88 | 9.9 | 157 | 89% | 319040 | 20091 |

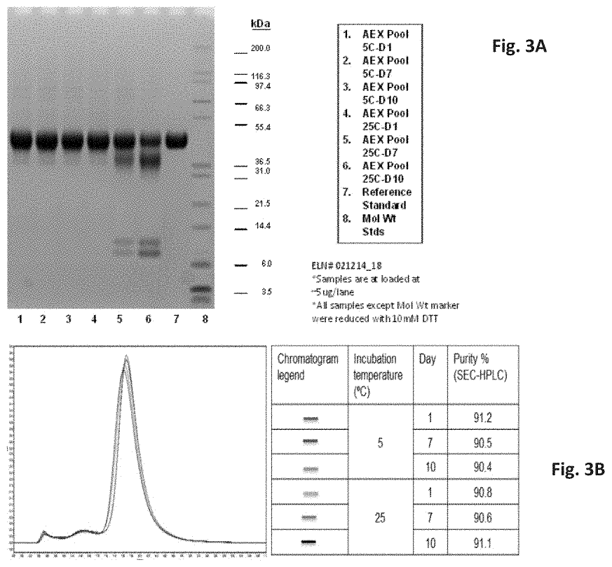

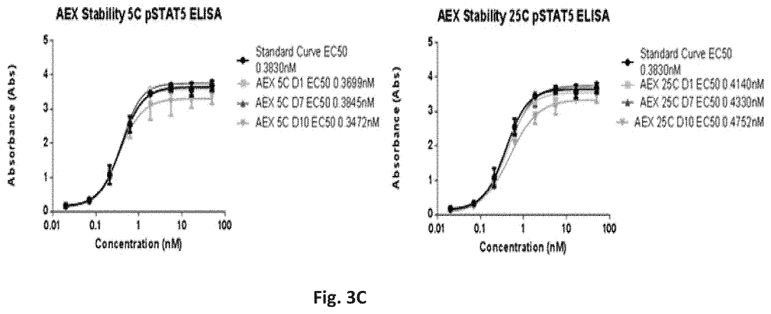

The stability and activity of Polypeptide A was determined after the AEX I step after storage at 5 and 25° C. for 1, 7, and 10 days. As shown by SDS-PAGE and SE-HPLC, Polypeptide A stability is maintained at days 1, 7, and 10 at 5° C. and day 1 at 25° C., although some degradation occurs at day 7 and 10 (FIG. 3A and FIG. 3B). Activity of Polypeptide A was measured in a cell-based assay. The indicator cell line, HH cells (ATCC: CRL-2105) is a human T-lymphocyte that lacks the IL-2Rα subunit of the IL-2 receptor. Polypeptide A has been engineered to more specifically bind to the lower affinity receptor IL-2β/γ which leads to the stimulation and phosphorylation of pSTAT5. The level of pSTAT5 activation in the HH cell line, in response to Polypeptide A, is then measured by a sandwich ELISA. The activity assay results show that Polypeptide A maintains activity at both temperatures over time (FIG. 3C).

### Protein Refolding/Elevated pH Hold

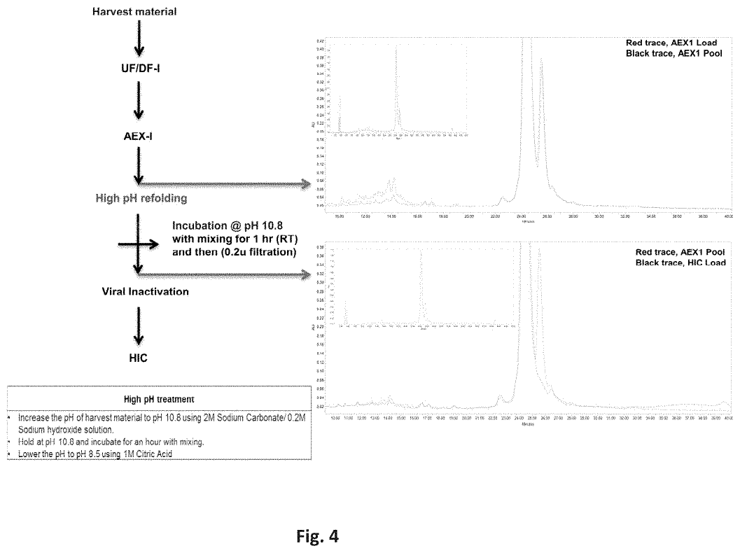

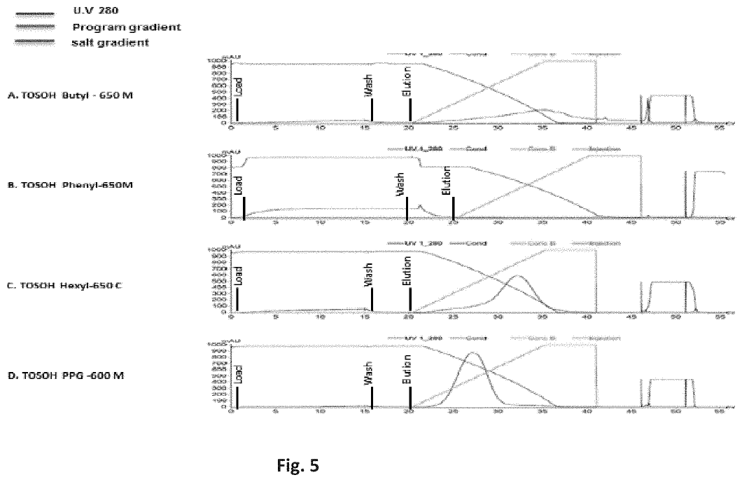

The AEX I product was then subjected to an elevated pH hold. A sodium carbonate (2 M)/sodium hydroxide (0.2 M) buffer was added to the AEX I pool to raise the pH to about 10.8. The AEX I pool was maintained at this pH for about 1 hour while mixing and ensuring the pH remained at ≥10.7. After the elevated pH incubation, a 1 M citric acid solution was added to the AEX I pool to decrease the pH to about 8.5+/−0.2. After the pH adjustment, the sample was filtered through a 0.2 μm filter. It was surprisingly discovered that the elevated pH hold after the AEX I step improved the purity of the product. The AEX I load sample and AEX I pool sample were analyzed by RP-HPLC before the elevated pH hold and the sample (HIC load) was analyzed by RP-HPLC after the elevated pH hold. As can be seen in FIG. 4, a minor peak, representing a contaminant, is seen to the right of the main peak corresponding to Polypeptide A. This minor peak is removed after the elevated pH hold step, as seen in the RP-HPLC trace for the HIC load sample.

### Viral Inactivation

The protein sample was subsequently subjected to a viral inactivation step. In the viral inactivation process, a solvent/detergent was used for virus inactivation of enveloped viruses. This method in particular has many advantages over other methods as it does not denature the protein, is compatible with most buffer system, has a high process recovery, and requires relatively simple equipment. The primary goal of this step is to inactivate any potential enveloped viruses that may be present in the process stream. The solvent and detergent used in this process was TnBP (Tri (n-butyl) phosphate) and sodium cholate. A 10× solution of the solvent and detergent was prepared at 3% TnBP and 10% sodium cholate. To inactivate the virus, the solvent/detergent was added to AEX column mainstream pool to a 1× final concentration, and the solution was mixed and incubated for 4 hours at ambient temperature. After the incubation was complete, 1M ammonium sulfate was added to prepare the mainstream pool for further downstream processing on the HIC column.

### Hydrophobic Interaction Chromatography (HIC)

HIC was the second purification step in the Polypeptide A downstream process. The resin matrix used in this chromatography step was Toyopearl PPG 600 M (Tosoh Biosciences) and has an average bead size of 40-90 μm. It was operated in a bind and elute mode at ambient temperature and used a multistep reverse gradient of ammonium sulfate to elute Polypeptide A.

### HIC Resin Screen

An initial screen was performed to determine the optimal HIC resin. During the initial resin screening, 1.5 M ammonium sulfate was added to the AEX column eluate pool and the pH adjusted to 8.0±0.2. This sample was then applied to different HIC resins to test the binding of Polypeptide A. Specifically, a Butyl-650 M, Phenyl-650 M, Hexyl-650 C, and PPG-600 M resin was tested. The columns were washed and a reverse linear gradient of decreasing salt concentration was applied to elute the bound Polypeptide A. Polypeptide A bound to Toyopearl PPG 600M resin, in the presence of 1.5 M ammonium sulfate at 8.0±0.2, and eluted with a reverse linear salt gradient of decreasing salt concentration. Based on the elution profile and SDS-PAGE analysis, Toyopearl PPG 600M was selected as the resin for further evaluation (FIG. 5).

A Toyopearl PPG 600M resin was used to generate a 20 cm packed resin bed with a DBC of 5 g/L. The HIC column was first equilibrated with alternating single column volumes of HIC buffer B (HIC B) and HIC buffer A (HIC A) for 4 total column volumes, at a flow rate of 179 cm/hr.

The AEX I step product was loaded onto the HIC column at a flow rate of 179 cm/hr. Following the loading step, the column was washed with HIC buffer A for 5 column volumes and a flow rate of 179 cm/hr.

Polypeptide A was then eluted from the HIC column with HIC buffer D (HIC D) with 10 column volumes and a flow rate of 179 cm/hr. Elutions were collected with an elution peak cutoff of 25 mAU. Table 3 below recites the HIC buffers used. Table 4 below shows a comparison of the HIC load to the HIC pooled elution sample. Polypeptide A amount, step yield, and CHO Host Cell Protein (HCP) amount are shown. All fractions collected during the elution step were analyzed by SE-HPLC and RP-HPLC for purity and concentration, respectively. Fractions whose purity was ≥95% by SE-HPLC were combined into a HIC pool for further processing.

TABLE 3
HIC buffers

| Buffer | Buffer | | Conductivity | |
|---|---|---|---|---|
| name | composition | pH | (mS/cm.) | Function |
| HIC A | 20 mM Tris- | 8.5 +/− 0.2 | 155-160 or | Column EQ, |
| | HCl + 1.1M | | 134-154 as an | Load, and |
| | ammonium | | alternative | Wash |
| | sulfate | | | |
| HIC B | 20 mM Tris- | 8.5 +/− 0.2 | <2 | Column EQ, |
| | HCl | | | Strip |
| HIC D | 20 mM Tris- | 8.5 +/− 0.1 | 125-130 or | Column |
| | HCl + 0.85M | | 108-126 as an | Elution |
| | ammonium | | alternative | |
| | sulfate | | | |

TABLE 4
HIC purification step results

| | | | Poly- | Step | | |
|---|---|---|---|---|---|---|
| | Titer | Volume | peptide | yield | CHO HCP | |
| Description | (mg/mL) | (L) | A (g) | (%) | ng/mL | ppm |
| HIC Load | 10.52 | 11.42 | 120 | N/A | 247010 | 23480 |
| Frac. # 1 | 0.18 | 15.8 | 3 | | 5188 | 28822 |
| Frac. # 2 | 1.23 | 15.9 | 20 | | 4444 | 3613 |
| Frac. # 3 | 1.5 | 15.9 | 24 | | 3093 | 2062 |
| Frac. # 4 | 1.12 | 17.1 | 19 | | 1175 | 1049 |
| Frac. #5 | 0.79 | 15.9 | 13 | | 653 | 827 |
| Frac. # 6 | 0.54 | 15.9 | 9 | | 427 | 791 |
| Frac. #7 | 0.39 | 15.9 | 6 | | 362 | 928 |
| Frac. #8 | 0.28 | 15.9 | 4 | | 312 | 1114 |
| Frac. # 9 | 0.19 | 15.9 | 3 | | 287 | 1511 |
| Frac. # 10 | 0.15 | 17.1 | 3 | | 231 | 1540 |
| Frac. # 11 | 0.11 | 14.5 | 2 | | 204 | 1855 |
| Frac. # 12 | 0.09 | 17.1 | 2 | | 186 | 2067 |
| Frac. # 13 | 0.06 | 14.6 | 1 | | 149 | 2483 |
| Frac. # 14 | 0.05 | 12 | 1 | | 122 | 2440 |
| HIC pool | 0.6 | 160 | 96 | 80% | 1899 | 3165 |
| (Frac. # 2 | | | | | | |
| to 11) | | | | | | |

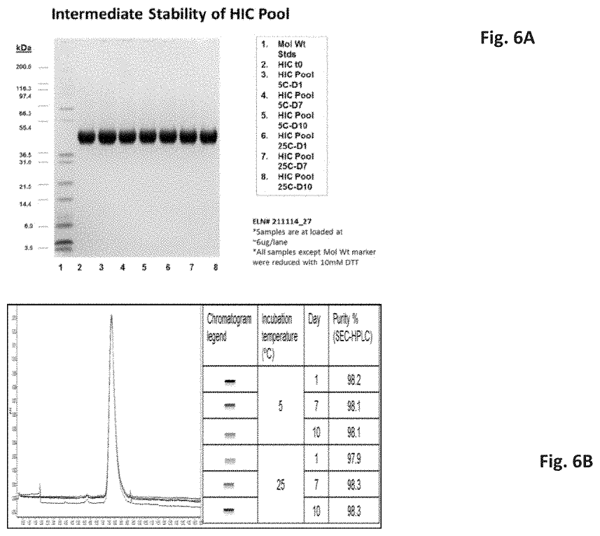

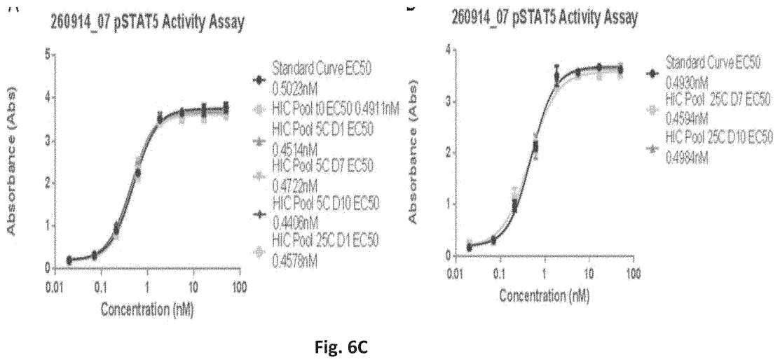

The stability and activity of Polypeptide A was determined after the HIC step after storage at 5 and 25° C. for 1, 7, and 10 days. As shown by SDS-PAGE and SE-HPLC, Polypeptide A stability is maintained at days 1, 7, and 10 at 5 and 25° C. (FIG. 6A and FIG. 6B). Activity of Polypeptide A was measured in the cell-based assay described previously. The activity assay results show that Polypeptide A maintains activity at both temperatures over time (FIG. 6C).

### UF/DF II

A second UF step was performed with a Sartorius Hydrosart 10 kDa MWCO filter having a membrane surface area of 1.8 m² (3×0.6 m²). The second UF step was used to concentration the product by a factor of about 10× or more. After concentration, the HIC pool was adjusted to pH 5.5 using glacial acetic acid, filtered through a 0.2 μm filter, and diafiltered into 50 mM sodium acetate, pH 5.5±0.2 buffer with a 3.0-4.0 mS conductivity for 5 DV. The final recovered product solution was tested via SE-HPLC and RP-HPLC for purity and concentration, respectively. Overall yield for this step was typically >90% and the product solution was stored between 2-8° C. until further processing.

### Mixed-Mode Chromatography (MMC)

MMC is the third step in the Polypeptide A downstream process. The resin selected for this chromatography step was Capto MMC ImpRes (G.E Healthcare), which is a weak cation exchange multimodal resin with an average particle size of 36-44 μm. It was operated in the bind and elute mode at ambient temperature to purify Polypeptide A. This step uses a multistep sodium chloride elution gradient designed to enhance resolution and reproducibility.

A Capto MMC ImpRes resin was used to generate a 20 cm packed resin bed with a DBC of 20 g/L. The MMC column was first equilibrated with alternating single column volumes of MMC buffer B (MMC B) and MMC buffer A (MMC A) for 4 total column volumes, at a flow rate of 238 cm/hr.

HIC step product was loaded onto the MMC column at a flow rate of 238 cm/hr. Following the loading step, the column was washed with MMC buffer A for 5 column volumes and a flow rate of 238 cm/hr followed by MMC buffer C (MMC C) for 5 column volumes and a flow rate of 238 cm/hr.

Polypeptide A was then eluted from the MMC column in a step elution process with MMC buffer D (MMC D) with 15 column volumes and a flow rate of 238 cm/hr. Elutions were collected with an elution peak cutoff of 50 mAU. An addition wash step was performed with MMC buffer E (MMC E) with 5 column volumes and a flow rate of 238 cm/hr. Table 5 below recites the MMC buffers used. Table 6 below shows a comparison of the MMC load to the MMC pooled elution sample. Polypeptide A amount, step yield, and CHO Host Cell Protein (HCP) amount are shown.

TABLE 5
MMC buffers

| Buffer | Buffer | | Conductivity | |
|---|---|---|---|---|
| name | composition | pH | (mS/cm.) | Function |
| MMC A | 50 mM NaOAc | 5.5 +/− 0.2 | 3-4 | Column EQ, |
| | | | | Load, and |
| | | | | Wash |
| MMC B | 50 mM | 5.5 +/− 0.2 | 84-92 | Column EQ, |
| | NaOAc + | | | Strip |
| | 1M NaCl | | | |
| MMC C | 50 mM | 5.5 +/− 0.2 | 24-30 | Column pre- |
| | NaOAc + | | | main peak |
| | 0.25M NaCl | | | elution |
| MMC D | 50 mM | 5.5 +/− 0.2 | 55-66 | Column main |
| | NaOAc + | | | peak elution |
| | 0.65M NaCl | | | |
| MMC E | 50 mM | 5.5 +/− 0.2 | 69-71 | Column post |
| | NaOAc + | | | main peak |
| | 0.70M NaCl | | | elution |

TABLE 6
MMC purification step results

| | | | Poly- | Step | | |
|---|---|---|---|---|---|---|
| | Titer | Volume | peptide | yield | CHO HCP | |
| Description | (mg/mL) | (L) | A (g) | (%) | ng/mL | ppm |
| MMC Load | 3.85 | 21.6 | 83 | N/A | 5831 | 1514 |
| Frac. # 1 | 1.95 | 7.6 | 15 | | 1427 | 732 |
| Frac. # 2 | 2.52 | 6.46 | 16 | | 312 | 124 |
| Frac. # 3 | 1.43 | 6.8 | 10 | | 70 | 49 |
| Frac. # 4 | 0.86 | 6.7 | 6 | | 44 | 51 |
| Frac. #5 | 0.79 | 7.4 | 6 | | 35 | 44 |
| Frac. # 6 | 0.57 | 6.85 | 4 | | 29 | 51 |
| Frac. #7 | 0.44 | 7 | 3 | | 23 | 52 |
| Frac. #8 | 0.34 | 7 | 2 | | 22 | 65 |
| Frac. # 9 | 0.28 | 7.1 | 2 | | 21 | 75 |
| Frac. # 10 | 0.22 | 6.9 | 2 | | 20 | 91 |
| Frac. # 11 | 0.18 | 7 | 1 | | 19 | 106 |
| Frac. # 12 | 0.14 | 7 | 1 | | 19 | 136 |
| Frac. # 13 | 0.12 | 7.4 | 1 | | 18 | 150 |
| Frac. # 14 | 0.08 | 7.3 | 1 | | 17 | 213 |
| Frac. # 15 | 0.08 | 3 | 0 | | 17 | 213 |
| Frac. # 16 | 0.08 | 6.3 | 1 | | 19 | 238 |
| Frac. # 17 | 0.1 | 7.2 | 1 | | 22 | 220 |
| MMC pool | N/A | 91.21 | 68 | 82% | 724 | 181 |
| (Frac. # 1 | | | | | | |
| to 13) | | | | | | |

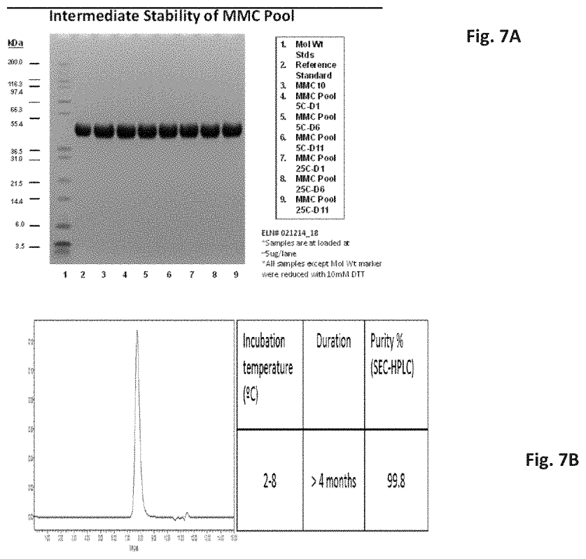

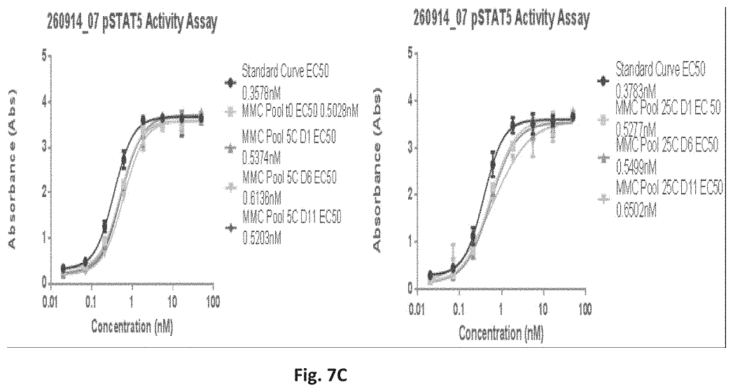

The stability and activity of Polypeptide A was determined after the MMC step after storage at 5 and 25° C. for 1, 6, and 11 days. As shown by SDS-PAGE, Polypeptide A stability is maintained at days 1, 6, and 11 at 5 and 25° C. (FIG. 7A). Polypeptide A stability is also maintained at 2-8° C. for 4 months, as shown by SE-HPLC (FIG. 7B). Activity of Polypeptide A was measured in the cell-based assay described previously. The activity assay results show that Polypeptide A maintains activity at both temperatures over time (FIG. 7C).

### UF/DF III

The third UF step was used to concentration the product by a factor of about 10× or more. After concentration, the MMC pool was diafiltered into 20 mM Tris, pH 8.0 buffer with a 2.0-3.0 mS conductivity for 5 DV.

### Anion Exchange Chromatography (AEX) II

Following the UF/DF III step, a second AEX step (AEX II) was employed as an additional polishing for Polypeptide A. The GigaBap Q 650 M resin (Tosoh Biosciences) was used with a 20 cm packed bed and a Dynamic Binding Capacity (DBC) of 20 g/L resin. The AEX column was first equilibrated with alternating single column volumes of AEX II buffer F (AEX F) and AEX II buffer E (AEX E) for 4 total column volumes, at a flow rate of 300 cm/hr.

The UF/DF III step product was loaded onto the AEX II column at a flow rate of 300 cm/hr. Following the loading step, the column was washed with AEX II buffer E for 5 column volumes and a flow rate of 300 cm/hr.

Polypeptide A was then eluted from the AEX II column with one of two different gradient elution steps. One gradient elution was performed going from AEX II buffer E to AEX II buffer F. The gradient with run at a flow rate of 300 cm/hr and went from 0-20% buffer E to buffer F over 15 column volumes. Elutions were collected with an elution peak cutoff of 200 mAU. Table 7 below recites the AEX II buffers used. Table 8 below shows a comparison of the AEX II load to the AEX II pooled elution sample. Polypeptide A amount, step yield, and CHO Host Cell Protein (HCP) amount are shown. The second alternative elution step was performed going from AEX II buffer E to AEX II buffer G. The gradient with run at a flow rate of 300 cm/hr and went from 0-100% buffer E to buffer G over 15 column volumes.

TABLE 7
AEX II buffers

| Buffer | Buffer | | Conductivity | |
|---|---|---|---|---|
| name | composition | pH | (mS/cm.) | Function |
| AEX E | 20 mM Tris- | 8.0 +/− 0.2 | ≤2 | Column EQ, |
| | HCl | | | Load, Wash, |
| | | | | and Elution |
| AEX F | 20 mM Tris- | 8.0 +/− 0.2 | 80-92 | Column EQ, |
| | HCl + 1M | | | Elution, and |
| | NaCl | | | Strip |
| AEX G | 20 mM Tris- | 8.0 +/− 0.2 | ~20 (18-24) | Elution |
| | HC1 + 0.2M | | | |
| | NaCl | | | |

TABLE 8
AEX II purification step results

| | | | Poly- | Step | | |
|---|---|---|---|---|---|---|
| | Titer | Volume | peptide | yield | CHO HCP | |
| Description | (mg/mL) | (L) | A (g) | (%) | ng/mL | ppm |
| AEX II | 4.01 | 17.8 | 71 | N/A | 724 | 181 |
| Load | | | | | | |
| Frac. # 1 | 0.49 | 3.1 | 2 | | 11 | 22 |
| Frac. # 2 | 1.77 | 3 | 5 | | 22 | 12 |
| Frac. # 3 | 2.35 | 3.1 | 7 | | 27 | 11 |
| Frac. # 4 | 2.49 | 3 | 7 | | 33 | 13 |
| Frac. #5 | 2.33 | 3.2 | 7 | | 38 | 16 |
| Frac. # 6 | 2.12 | 3.1 | 7 | | 41 | 19 |
| Frac. #7 | 1.9 | 3 | 6 | | 41 | 22 |
| Frac. #8 | 1.55 | 3 | 5 | | 42 | 27 |
| Frac. # 9 | 1.39 | 3.1 | 4 | | 46 | 33 |
| Frac. # 10 | 1.06 | 2.9 | 3 | | 55 | 52 |
| Frac. # 11 | 0.84 | 3 | 3 | | 62 | 74 |
| Frac. # 12 | 0.59 | 2.8 | 2 | | 75 | 127 |
| Frac. # 13 | 0.38 | 3.1 | 1 | | 111 | 292 |
| Frac. # 14 | 0.24 | 2.7 | 1 | | 172 | 717 |
| AEX II pool | 1.81 | 33.5 | 61 | 85% | 29 | 23 |
| (Frac. # 1 | | | | | | |
| to 11) | | | | | | |

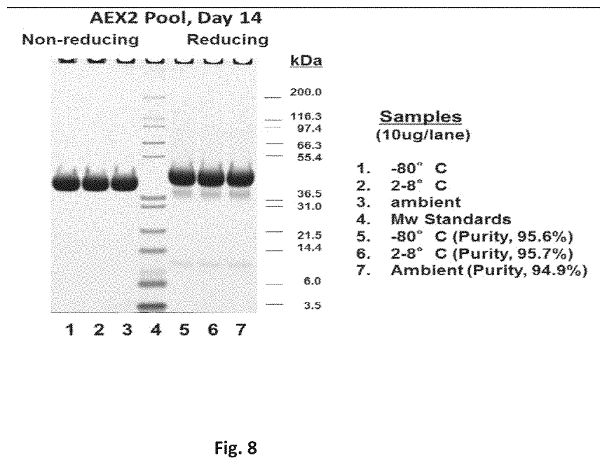

The stability of Polypeptide A was determined after the AEX II step after storage at 2-8, 25, and −80° C. for 14 days. As shown by SDS-PAGE, Polypeptide A stability is maintained 14 days at 2-8, 25, and −80° C. (FIG. 8).

### Reduction of Host Cell Protein (HCP) Content

HCP content in a pharmaceutical composition may increase the risk of immunogenicity when administered to a patient. Reduction of the HCP content has been linked to a reduction in specific inflammatory cytokines (Wang et al. Biotechnology & Bioengineering. 103(3): 446-58. 2009). It is therefore advantageous to reduce HCP content in a pharmaceutical composition as low as possible. Despite three chromatography steps (AEX I, HIC, and MMC) and several filtration steps, the HCP content in the Polypeptide A purification remained above 150 ppm. It was surprisingly discovered that the above recited AEX II step was important to reduce HCP content to a lower level, e.g., to a HCP content of ≤ about 100 μm or ≤ about 50 ppm. In particular, the use of a gradient elution step, rather than the single-buffer elution of the AEX I step, was useful in reducing the HCP content to ≤ about 100 μm. The final AEX II step allowed for the reduction of HCP content to acceptable levels without the need for an affinity purification step.

### Viral Filtration

The AEX II pool sample was then subjected to a viral filtration step using EMD Viresolve Pro Modus 1.3 Shield Prefilter (1.3 inches) and Device (0.22 m²). The filtration step was performed at a flow rate of 2600 mL/min (990-2750 mL/min range).

### UF/DF IV and Bulk Drug Fill

The fourth UF step was used to concentration the product by a factor of about 10× or more. After concentration, the AEX II pool was diafiltered into the formulation buffer (50 mg/ml sucrose, 2.03 mg/ml sodium citrate tribasic dihydrate, and 0.97 mg/ml citric acid, pH 6.1) for 10 DV. Polysorbate 20 was then added to the diafiltered sample to a final concentration of 0.1 mg/ml. The final concentration of Polypeptide A was brought to 1.05 mg/mL (1.0-1.09 mg/mL range). Finally, the sample was filtered through a 0.1 um Millipore Durapore filter.

### Purification Summary

Table 9 below summarize the Polypeptide A titer and HCP contaminant results of the above recited purification scheme.

TABLE 9
Reverse Phase (RP)-HPLC determined Polypeptide
A titer and CHO Host Cell Protein (HCP) in samples
at different stages of purification scheme

| | |
|---|---|
| RP-HPLC Titer | CHO HCP |

| Sample | (mg/mL) | ng/mL | ppm |
|---|---|---|---|
| AEX 1 Load | 11.36 | 789380 | 69488 |
| AEX I Pool | 15.88 | 319040 | 20091 |
| HIC Load | 10.52 | 247010 | 23480 |
| HIC Pool | 0.60 | 1899 | 3165 |
| MMC Load | 3.85 | 5831 | 1515 |
| AEX 2 Load | 4.01 | 724 | 181 |
| AEX 2 Pool | 1.28 | 29 | 23 |

### Example 3—Optimization of pH Hold Step to Improve Purity

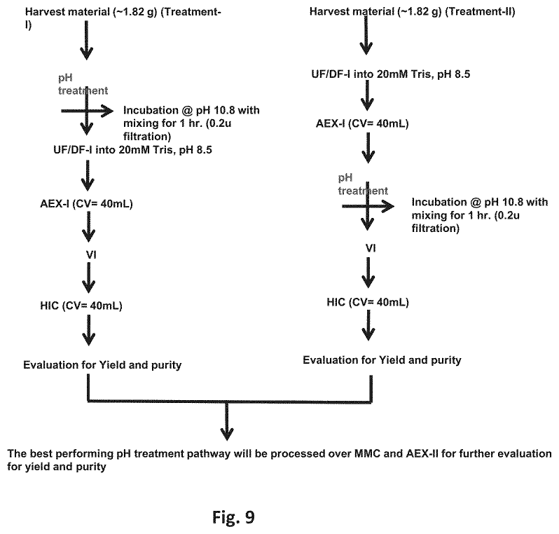

In an effort to improve purity of Polypeptide A during the purification process, the protein refolding/elevated pH hold step was optimized. The results of this Example led to the protein refolding/elevated pH hold step as described above in Example 2. The 1 hour hold at a pH of 10.8 was performed before the AEX I step or after the AEX I step and the results were compared. A schematic of the two experiments performed is depicted in FIG. 9.

### pH Treatment Before AEX I

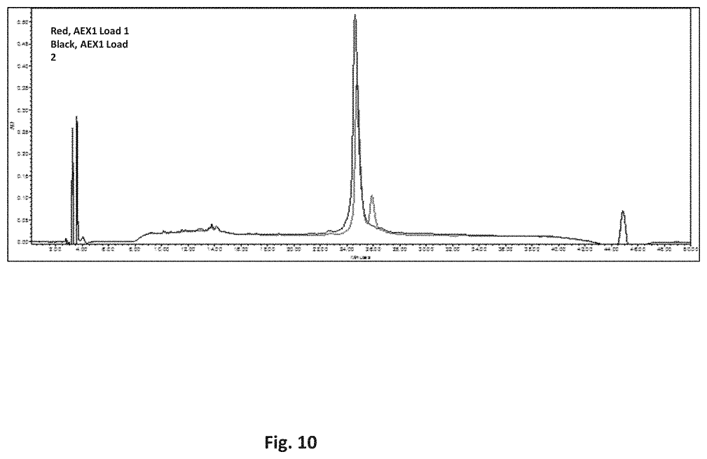

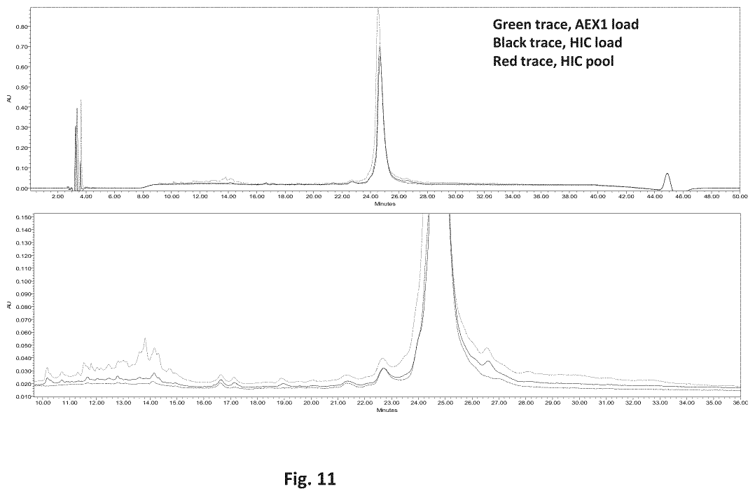

The harvest material prior to the UF/DF I and AEX I step was incubated at pH 10.8 for 1 hour with mixing. The pH treated sample was then processed as described in Example 2 with the UF/DF I step, the AEX I step, the viral inactivation step, and HIC step. The results of the AEX I step are shown in Table 10 below and FIG. 10. The results of the subsequent HIC step are shown in Table 11 below and FIG. 11.

TABLE 10
Results of AEX I step from pH treated harvest sample

| | | | Poly- | | | |
|---|---|---|---|---|---|---|
| | Titer | Volume | peptide | Yield | CHO HCP | |
| Sample | (mg/mL) | (mL) | A (mg) | (%) | ng/mL | ppm |
| AEX I | 5.46 | 218 | 1190 | n/a | 1107300 | 202802 |
| Load 2 | | | | | | |
| AEX I | 29.37 | 20 | 587 | 49 | 1156700 | 39384 |
| Frac- | | | | | | |
| tion 1 | | | | | | |
| AEX I | 11.33 | 20 | 227 | 19 | 1359100 | 119956 |
| Frac- | | | | | | |
| tion 2 | | | | | | |
| AEX I | 4.47 | 20 | 89 | 8 | 963200 | 215481 |
| Frac- | | | | | | |
| tion 3 | | | | | | |
| AEX I | 2.29 | 20 | 46 | 4 | 902900 | 394279 |
| Frac- | | | | | | |
| tion 4 | | | | | | |
| AEX I | 1.25 | 20 | 25 | 2 | 713800 | 571010 |
| Frac- | | | | | | |
| tion 5 | | | | | | |
| AEX I | 0.86 | 9.5 | 8 | 1 | 478500 | 556395 |
| Frac- | | | | | | |
| tion 6 | | | | | | |
| | | | Overall | 83 | Not | Not |
| | | | yield | | deter- | deter- |
| | | | | | mined | mined |
| | | | Frac. | 68 | 79670 | 2.5 fold |
| | | | 1 and 2 | | | |
| | | | Frac. | 76 | 124940 | 1.6 fold |
| | | | 1-3 | | | |

TABLE 11
Results of HIC step from pH treated harvest sample

| | | | Poly- | | | | Clear- |
|---|---|---|---|---|---|---|---|
| Sam- | Titer | Volume | peptide | CHO HCP | | Yield | ance |
| ple | (mg/mL) | (mL) | A (mg) | ng/mL | ppm | % | Fold |
| HIC | 7.49 | 27 | 202 | 731060 | 97605 | N/A | |
| Load | | | | | | | |
| HIC | 0.08 | 20 | 1.6 | 15909 | 198863 | | |
| Frac- | | | | | | | |
| tion 1 | | | | | | | |
| HIC | 0.46 | 20 | 9.2 | 14667 | 31885 | | |
| Frac- | | | | | | | |
| tion 2 | | | | | | | |
| HIC | 1.05 | 20 | 21 | 11287 | 10750 | | |
| Frac- | | | | | | | |
| tion 3 | | | | | | | |
| HIC | 0.85 | 20 | 17 | 5407 | 6361 | | |
| Frac- | | | | | | | |
| tion 4 | | | | | | | |
| HIC | 0.54 | 20 | 10.8 | 4309 | 7980 | | |
| Frac- | | | | | | | |
| tion 5 | | | | | | | |
| HIC | 0.25 | 20 | 5 | 3249 | 12996 | | |
| Frac- | | | | | | | |
| tion 6 | | | | | | | |
| HIC | 0.58 | 220 | 127.6 | 9377 | 16167 | 63 | 6 |
| Pool | | | | | | | |
| (Frac. | | | | | | | |
| 1-7) | | | | | | | |

pH Treatment after AEX I

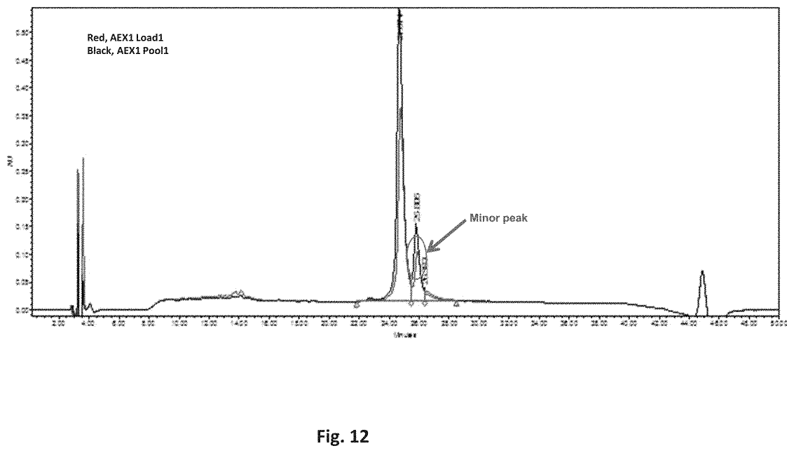

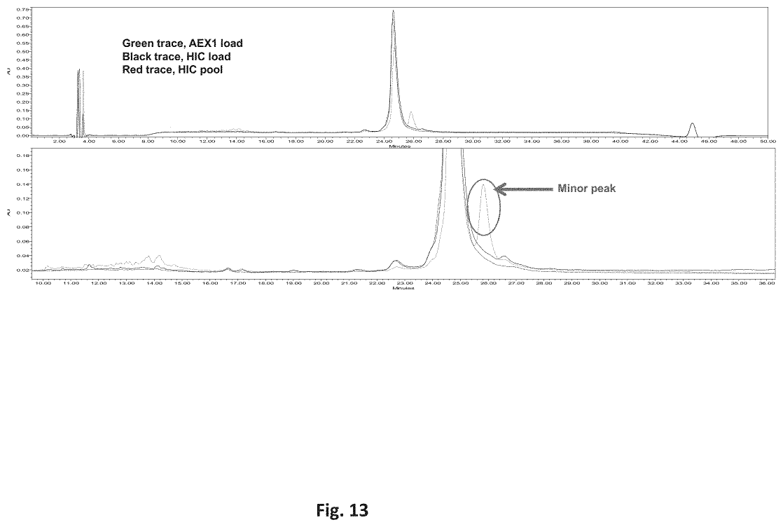

The material obtained after the UF/DF I and AEX I step was incubated at pH 10.8 for 1 hour with mixing. The pH treated sample was then processed as described in Example 2 with the viral inactivation step and HIC step. The results of the AEX I step are shown in Table 12 below and FIG. 12. The results of the subsequent HIC step are shown in Table 13 below and FIG. 13.

TABLE 12
Results of AEX I step before pH treatment

| | | | Poly- | | | |
|---|---|---|---|---|---|---|
| | Titer | Volume | peptide | Yield | CHO HCP | |
| Sample | (mg/mL) | (mL) | A (mg) | (%) | ng/mL | ppm |
| AEX I | 4.15 | 285 | 1183 | n/a | 762900 | 183831 |
| Load 1 | | | | | | |
| AEX I | 18.73 | 54 | 1011 | 86 | 954000 | 50934 |
| Pool 1 | | | | | | |

4 fold

TABLE 13
Results of HIC step from pH treated AEX I sample

| | | Poly- | | | | Clear- |
|---|---|---|---|---|---|---|
| Sam- | Titer | peptide | CHO HCP | | Yield | ance |
| ple | (mg/mL) | A (mg) | ng/mL | ppm | % | Fold |
| HIC | 7.95 | 174.9 | 483730 | 60847 | N/A | |
| Load | | | | | | |
| HIC | 0.10 | 2 | 15646 | 156460 | | |
| Frac- | | | | | | |
| tion 1 | | | | | | |
| HIC | 0.48 | 9.6 | 10511 | 21898 | | |
| Frac- | | | | | | |
| tion 2 | | | | | | |
| HIC | 1.02 | 20.4 | 4763 | 4670 | | |
| Frac- | | | | | | |
| tion 3 | | | | | | |
| HIC | 0.66 | 13.2 | 3428 | 5194 | | |
| Frac- | | | | | | |
| tion 4 | | | | | | |
| HIC | 0.43 | 8.6 | 3126 | 7270 | | |
| Frac- | | | | | | |
| tion 5 | | | | | | |
| HIC | 0.29 | 5.8 | 2714 | 9359 | | |
| Frac- | | | | | | |
| tion 6 | | | | | | |
| HIC | 0.21 | 4.2 | 2348 | 11181 | | |
| Frac- | | | | | | |
| tion 7 | | | | | | |
| HIC | 0.15 | 3 | 2043 | 1362 | | |
| Frac- | | | | | | |
| tion 8 | | | | | | |
| HIC | 0.56 | 123.2 | 5762 | 10289 | 70 | 6 |
| Pool | | | | | | |
| (Frac. | | | | | | |
| 1-7) | | | | | | |

As can be seen from the data described above, the AEX I column does not resolve away the minor peak contaminant. However, as shown in FIG. 13, the minor peak is not observed when the AEX I pool sample is subjected to the elevated pH treatment.

### Example 4—Site-Specific Glycan Profiling of Polypeptide A Following Purification

The site-specific glycan profile of Polypeptide A was determined following the downstream purification process described in Example 2. The purified composition contains a mixture of Polypeptide A polypeptides with different glycan structures on select amino acids. To determine the glycan profile, peptide mapping was performed. Briefly, reduced and alkylated Polypeptide A protein was incompletely digested using the protease trypsin and the digestion mixture separated using reverse phase high performance liquid chromatography mass spectrometry (RP-HPLC/MS). This yielded a large set of overlapping peptides and the analysis confirmed the theoretical sequence. A total ion chromatogram of the Trypsin-generated Peptide Map for three different lots of purified Polypeptide A confirmed that the lots were comparable. N-linked glycans were identified as being present at Asn187 (N187) and Asn206 (N206). These glycans were predominantly sialylated, complex fucosylated types. Core 1 type 0-glycans were also present at Thr212 (T212).

The relative percentage of the glycan isoform at each glycosylation site was calculated based on the ion intensity and the results are summarized below in Table 14. The glycan profiles are highly similar among the three lots, showing the same major glycan species for each of the lots.

TABLE 14
Site-Specific Glycan Profiling of Polypeptide A

| | | | Relative | | |
|---|---|---|---|---|---|
| | | | Percent (%) | | |
| Residue | Sequence | Glycan Form | Lot 1 | Lot 2 | Lot 3 |
| 177- | SGSLYMLCTGN₁₈₇S | Hex5HexNAc4FucNeuAc2 | 67.72 | 63.79 | 63.69 |
| 205 | SHSSWDNQCQCTSS | Hex6HexNAc5FucNeuAc2 | 4.20 | 5.84 | 5.90 |
| | ATR | Hex5HexNAc4FucNeuAc | 7.49 | 8.88 | 8.89 |
| | (SEQ ID NO: 2) | Hex6HexNAc5FucNeuAc3 | 15.29 | 16.57 | 16.31 |
| | | Hex4HexNAc4FucNeuAc | 1.04 | 0.55 | 0.59 |
| | | Hex5HexNAc5NeuAc2 | 3.80 | 3.63 | 3.86 |
| | | Hex5HexNAc4Fuc | 0.18 | 0.33 | 0.33 |
| | | Hex3HexNAc4Fuc | 0.09 | 0.15 | 0.13 |
| | | Hex4HexNAc4Fuc | 0.15 | 0.18 | 0.20 |
| | | Hex6HexNAc5Fuc | 0.04 | 0.06 | 0.07 |
| | | Hex5HexNAc5Fuc | 0.03 | 0.03 | 0.04 |
| 206- | N₂₀₆TTK | Hex6HexNAc5FucNeuAc3 | 4.44 | 4.39 | 3.92 |
| 209 | (SEQ ID NO: 3) | Hex5HexNAc4FucNeuAc2 | 82.92 | 77.79 | 76.65 |
| | | Hex6HexNAc5FucNeuAc2 | 2.71 | 3.91 | 3.69 |
| | | Hex7HexNAc6FucNeuAc3 | 0.49 | 0.71 | 0.53 |
| | | Hex6HexNAc5FucNeuAc | 0.21 | 0.74 | 0.88 |
| | | Hex5HexNAc4FucNeuAc | 6.79 | 9.40 | 10.68 |
| | | Hex5HexNAc4Fuc | 1.39 | 2.11 | 2.11 |
| | | Hex4HexNAc4Fuc | 1.04 | 0.94 | 1.52 |
| 210- | QVT₂₁₂PQPEEQKER | HexHexNAc | 0.18 | 0.21 | 0.27 |
| 221 | (SEQ ID NO: 4) | HexHexNAcNeuAc | 15.56 | 17.69 | 17.75 |
| | | HexHexNAcNeuAc2 | 9.15 | 10.99 | 12.05 |
| | | Non-glycosylated | 75.11 | 71.11 | 69.94 |

Hex: Hexose;
HexNAc: N-acetylhexosamine;
NeuAc: N-acetylneuraminic acid;
Fuc: Fucose

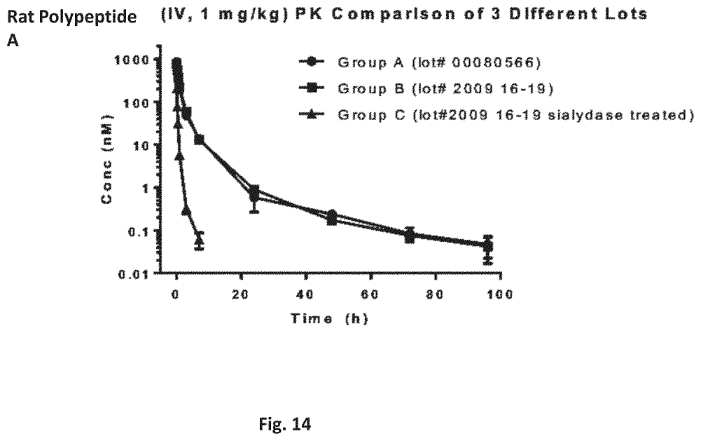

To demonstrate the importance of the glycan structures on the purified Polypeptide A, three different lots of purified Polypeptide A were used in a pharmacokinetic assay in mice to determine serum half-life. One of the three lots was treated with a sialidase to remove sialyation marks, while the other two lots were untreated. As demonstrated in FIG. 14, removal of the sialyation marks greatly diminished the serum half-life of Polypeptide A. The downstream purification process described in Example 2 leads to a purified composition containing a mixture of Polypeptide A polypeptides with different glycan structures on select amino acids. The glycan structures are shown here to be important in maintaining serum half-life.

### Example 5—Charge Distribution of Polypeptide A Following Purification

The charge distribution profile was determined following the downstream purification process recited in Example 2. The purified composition contains a mixture of Polypeptide A polypeptides with different charges. To determine the charge distribution profile, three different lots of purified Polypeptide A were analyzed with capillary isoelectric focusing (cIEF) to generate cIEF profiles. The pI peak area percentage was then measured to determine relative amounts of each charge variant in the purified Polypeptide A composition. The results are depicted below in Table 15. The cIEF profiles of the three lots are depicted in FIG. 15.

TABLE 15
cIEF Peak Area Percent for Charge Variants of Polypeptide A

| | Lot 1 | Lot 2 | Lot 3 |
|---|---|---|---|
| pI | (%) | (%) | (%) |
| 5.73 | 1.7 | 2.2 | 2.3 |
| 5.93 | 8.9 | 11.6 | 11.5 |
| 6.09 | 20.9 | 24.7 | 24.9 |
| 6.28 | 3.8 | 4.0 | 4.1 |
| 6.38 | 23.2 | 25.0 | 25.0 |
| 6.48 | 3.6 | 3.6 | 3.8 |
| 6.53 | 4.6 | 4.8 | 4.7 |
| 6.66 | 26.4 | 20.7 | 20.6 |
| 6.82 | 4.8 | 3.4 | 3.1 |
| 7.02 | 2.2 | None detected | None detected |
| 5.73a | 1.7 | 2.2 | 2.3 |
| 5.93b | 8.9 | 11.6 | 11.5 |

pI 5.73 is the combined peaks of pI 5.70 and pI 5.76.
pI 5.93 is the combined peaks of pI 5.89 and pI 5.97.

1. A method of purifying a polypeptide comprising a circularly permuted IL-2 fused to the extracellular portion of an IL-2Rα chain, the method comprising:
a) contacting a clarified cell supernatant comprising the polypeptide and host cell protein (HCP) with a first chromatography matrix under conditions such that the polypeptide binds to the matrix, and selectively eluting the polypeptide from the matrix in a first eluate;
b) adjusting the pH of the first eluate from step (a) to at least 10.5 and at most 11.5 to produce a pH-adjusted eluate; and
c) contacting the pH-adjusted eluate from step (b) with a second chromatography matrix such that the polypeptide binds to the matrix, and selectively eluting the polypeptide from the matrix in a second eluate, thereby purifying the polypeptide.

2. The method of claim 1, wherein the polypeptide comprises: an amino acid sequence that is at least 85%, 90%, 95%, or 99% identical to SEQ ID NO: 1; or that comprises the amino acid sequence of SEQ ID NO: 1.

3. The method of claim 1, wherein the first chromatography matrix comprises an anion exchange chromatography (AEX) matrix, optionally wherein:
prior to contact with the AEX matrix the clarified cell supernatant is buffer-exchanged into a solution having a conductivity of about 1-2 mS/cm and a pH of about 8.0-8.5;
the AEX matrix comprises quaternary amine groups; and/or
the AEX matrix comprises hydroxylated methacrylic polymer beads functionalized with quaternary amine groups, optionally wherein the mean diameter of the beads is about 75 μm and the mean pore size of the beads is about 100 nm.

4-8. (canceled)

9. The method of claim 3, wherein:
the polypeptide is eluted from the AEX matrix using a solution having a salt concentration equivalent of conductivity of about 15 to about 25 mS/cm; and/or
the polypeptide is eluted from the AEX matrix using an aqueous solution of about 0.20-0.25 M sodium chloride at about pH 8.4-8.6.

10. (canceled)

11. The method of claim 1, wherein:
the pH of the first eluate is adjusted using sodium carbonate and/or sodium hydroxide;
the pH of the first eluate is adjusted using sodium carbonate or sodium hydroxide at a ratio of about 0.1 kg sodium carbonate or sodium hydroxide to about 1 kg of the first eluate;
the pH of the pH-adjusted eluate is maintained at or above 10.7 but below 11.0 for at least 1 hour;
the pH of the pH-adjusted eluate is maintained at or above 10.7 but below 11.0 for at least 1 hour, wherein the pH at or above 10.7 but below 11.0 is achieved by adding sodium carbonate or sodium hydroxide at a ratio of about 0.1 kg sodium carbonate or sodium hydroxide to about 1 kg of the first eluate;
the method further comprises lowering the pH of the pH-adjusted eluate by adding citric acid;
the method further comprises lowering the pH of the pH-adjusted eluate to about pH 8.3-8.7; and/or
prior to contact with a HIC matrix the conductivity of the pH-adjusted eluate is altered to be about 130 to about 160 mS/cm and the pH is altered to be about 8.5±0.2, optionally wherein the conductivity of the pH-adjusted eluate is altered using ammonium sulfate.

12-18. (canceled)

19. The method of claim 1, wherein the second chromatography matrix comprises a hydrophobic interaction chromatography (HIC) matrix, optionally wherein:
prior to contact with the HIC matrix the pH-adjusted eluate is altered to comprise about 1-1.2 M ammonium sulfate;
the HIC matrix comprises polypropylene glycol groups:
the HIC matrix comprises hydroxylated methacrylic polymer beads linked to polypropylene glycol group, optionally wherein the mean diameter of the beads is about 40 to about 90 μm and the mean pore size of the beads is about 75 nm;
the polypeptide is eluted from the HIC matrix using a solution having a salt concentration equivalent of conductivity of about 100 to about 140 mS/cm;
the polypeptide is eluted from the HIC matrix using a sequential multistep gradient of decreasing salt concentration;
the polypeptide is eluted from the HIC matrix using a buffer comprising about 0.85 to about 0.95 M ammonium sulfate at pH 8.5±0.2;
prior to contact with the HIC matrix the pH-adjusted eluate is filtered through a 0.2 μm filter; and/or
prior to contact with the second chromatography matrix the pH-adjusted eluate is subject to viral inactivation, optionally wherein the viral inactivation is achieved by admixture of the pH-adjusted eluate with tri-n-butyl phosphate and polysorbate 20.

20-30. (canceled)

31. The method of claim 1, wherein the polypeptide is further purified from the second eluate using mixed-mode chromatography (MMC), optionally wherein:
the polypeptide is further purified from the second eluate using MMC followed by AEX;
the clarified cell supernatant is from a Chinese hamster ovary (CHO) cell culture; and/or
the polypeptide in the second eluate is at least 90% pure, optionally wherein purity of the polypeptide is determined by Reverse Phase (RP) HPLC.

32-35. (canceled)

36. A method for reducing host cell protein (HCP) content from a clarified cell supernatant containing a polypeptide comprising a circularly permuted IL-2 fused to the extracellular portion of an IL-2Rα chain, the method comprising contacting a partially purified polypeptide with an AEX matrix under conditions such that the polypeptide binds to the AEX matrix, and selectively eluting the polypeptide from the AEX matrix under gradient elution conditions, thereby reducing HCP content from the polypeptide.

37. The method of claim 36, wherein the polypeptide comprises: an amino acid sequence that is at least 85%, 90%, 95%, or 99% identical to SEQ ID NO: 1; or that comprises the amino acid sequence of SEQ ID NO: 1.

38. The method of claim 36, wherein:
the method further comprises contacting the clarified cell supernatant comprising the polypeptide and HCP with one or more chromatography resins to obtain the partially purified polypeptide;
the HCP content is ≥ about 300 ppm or ≥ about 150 ppm prior to contacting the partially purified polypeptide with the AEX matrix; and/or
the HCP content is ≤ about 100 ppm or ≤ about 50 ppm after eluting the polypeptide from the AEX matrix under gradient elution conditions.

39-42. (canceled)

43. The method of claim 36, wherein the gradient elution conditions comprise one or more of:
increasing the conductivity of an elution buffer over time;
increasing the salt concentration of an elution buffer over time; or
decreasing the pH of an elution buffer over time, optionally wherein:
the conductivity of the elution buffer is increased from about ≤5 mS/cm to about ≥15 mS/cm, about ≤2 mS/cm to about ≥15 mS/cm, or about ≤2 mS/cm to about ≥20 mS/cm;
the conductivity of the elution buffer is increased to between about 15 mS/cm to about 100 mS/cm or about 20 mS/cm to about 50 mS/cm;
the elution buffer comprises a final conductivity of between about 20 mS/cm to about 50 mS/cm;
the elution buffer initially comprises a conductivity of about ≤5 mS/cm or about ≤2 mS/cm;
the salt concentration of the elution buffer is increased from about 0 M salt to about 1.5 M salt, about 0 M salt to about 1.0 M salt, about 0 M salt to about 0.5 M salt, or about 0.2 M salt to about 1.0 M salt;
the elution buffer comprises a final salt concentration of about 0.2 M salt;
the salt comprises sodium chloride;
the elution buffer further comprises a pH of about 7.0 to about 9.0 or about 8.0; and/or
the one or more chromatography resins to obtain the partially purified polypeptide are selected from the group consisting of AEX, HIC, and MMC.

44-60. (canceled)

61. The method of claim 43, wherein obtaining the partially purified polypeptide comprises the steps of:
a1) contacting the clarified cell supernatant comprising the polypeptide and HCP with a first AEX matrix under conditions such that the polypeptide binds to the AEX matrix, and selectively eluting the polypeptide from the AEX matrix in a first eluate;
a2) contacting the first eluate from step (a1) with a HIC matrix such that the polypeptide binds to the HIC matrix, and selectively eluting the polypeptide from the HIC matrix in a second eluate; and
a3) contacting the second eluate from step (a2) with a MMC matrix such that the polypeptide binds to the MMC matrix, and selectively eluting the polypeptide from the MMC matrix in a third eluate, thereby obtaining the partially purified polypeptide optionally wherein:
prior to contact with the first AEX matrix the clarified cell supernatant is buffer-exchanged into a solution having a conductivity of about 1-2 mS/cm and a pH of about 8.0-8.5;
the first AEX matrix comprises quaternary amine groups;
the first AEX matrix comprises hydroxylated methacrylic polymer beads functionalized with quaternary amine groups, optionally wherein the mean diameter of the beads is about 75 μm and the mean pore size of the beads is about 100 nm;
the polypeptide is eluted from the first AEX matrix using a solution having a salt concentration equivalent of conductivity of about 15 to about 25 mS/cm;
the polypeptide is eluted from the first AEX matrix using an aqueous solution of about 0.20-0.25 M sodium chloride at about pH 8.4-8.6;
the pH of the first eluate is adjusted using sodium carbonate and/or sodium hydroxide to produce a pH-adjusted first eluate, optionally wherein the pH of the pH-adjusted first eluate is maintained at or above 10.7 but below 11.0 for at least 1 hour;
the pH of the first eluate is adjusted using sodium carbonate or sodium hydroxide at a ratio of about 0.1 kg sodium carbonate or sodium hydroxide to about 1 kg of the first eluate;
the pH of the pH-adjusted eluate is maintained at or above 10.7 but below 11.0 for at least 1 hour, wherein the pH at or above 10.7 but below 11.0 is achieved by adding sodium carbonate or sodium hydroxide at a ratio of about 0.1 kg sodium carbonate or sodium hydroxide to about 1 kg of the first eluate;
the method further comprises lowering the pH of the pH-adjusted first eluate by adding citric acid;
prior to contact with the HIC matrix the conductivity of the pH-adjusted first eluate is altered to be about 130 to about 160 mS/cm and the pH is altered to be about 8.5±0.2, optionally the conductivity of the pH-adjusted first eluate is altered using ammonium sulfate;
prior to contact with the HIC matrix the pH-adjusted first eluate is altered to comprise about 1-1.2 M ammonium sulfate;
the HIC matrix comprises polypropylene glycol groups;
the HIC matrix comprises hydroxylated methacrylic polymer beads linked to polypropylene glycol groups optionally wherein the mean diameter of the beads is about 40 to about 90 μm and wherein the mean pore size of the beads is about 75 nm;
the polypeptide is eluted from the HIC matrix using a solution having a salt concentration equivalent of conductivity of about 100 to about 140 mS/cm;
the polypeptide is eluted from the HIC matrix using a sequential multistep gradient of decreasing salt concentration;
the polypeptide is eluted from the HIC matrix using a buffer comprising about 0.85 to about 0.95 M ammonium sulfate at pH 8.5±0.2;
prior to contact with the HIC matrix the pH-adjusted eluate is filtered through a 0.2 μm filter;
prior to contact with the HIC matrix the pH-adjusted first eluate is subject to viral inactivation, optionally wherein the viral inactivation is achieved by admixture of the pH-adjusted eluate with tri-n-butyl phosphate and polysorbate 20;
the clarified cell supernatant is from a Chinese hamster ovary (CHO) cell culture; and/or
an affinity purification step is not used.

62-88. (canceled)

89. A method for reducing host cell protein (HCP) content from a clarified cell supernatant to containing a polypeptide comprising a circularly permuted IL-2 fused to the extracellular portion of an IL-2Rα chain, the method comprising the steps of:
a. contacting the clarified cell supernatant comprising the polypeptide and HCP with a first AEX matrix under conditions such that the polypeptide binds to the first AEX matrix, and selectively eluting the polypeptide from the first AEX matrix in a first eluate;
b. contacting the first eluate with a HIC matrix such that the polypeptide binds to the HIC matrix, and selectively eluting the polypeptide from the HIC matrix in a second eluate;
c. contacting the second eluate with a MMC matrix such that the polypeptide binds to the MMC matrix, and selectively eluting the polypeptide from the MMC matrix in a third eluate; and
d. contacting the third eluate with a second AEX matrix under conditions such that the polypeptide binds to the second AEX matrix, and selectively eluting the polypeptide from the second AEX matrix under gradient elution conditions, thereby reducing HCP content from the polypeptide, wherein the HCP content is ≤ about 50 ppm after step (d),
optionally wherein the polypeptide comprises: an amino acid sequence that is at least 85%, 90%, 95%, or 99% identical to SEQ ID NO: 1, or that comprises the amino acid sequence of SEQ ID NO: 1.

90-91. (canceled)

92. A composition comprising a polypeptide comprising a circularly permuted IL-2 fused to the extracellular portion of an IL-2Rα chain, wherein the HCP content of the composition comprises ≤ about 100 ppm or about ≤ about 50 ppm, optionally wherein the polypeptide comprises: an amino acid sequence that is at least 85%, 90%, 95%, or 99% identical to SEQ ID NO: 1, or that comprises the amino acid sequence of SEQ ID NO: 1.

93-94. (canceled)

95. A composition comprising a polypeptide comprising a circularly permuted IL-2 fused to the extracellular portion of an IL-2Rα chain, wherein the HCP content of the composition comprises ≤ about 100 ppm, produced by the method of claim 36.

96. A method of improving the serum half-life of a composition comprising a plurality of polypeptides, each polypeptide of the plurality comprising a circularly permuted IL-2 fused to the extracellular portion of an IL-2Rα chain, the method comprising the steps of:
a. contacting the clarified cell supernatant comprising the polypeptide with a first AEX matrix under conditions such that the polypeptide binds to the first AEX matrix, and selectively eluting the polypeptide from the first AEX matrix in a first eluate;
b. contacting the first eluate with a HIC matrix such that the polypeptide binds to the HIC matrix, and selectively eluting the polypeptide from the HIC matrix in a second eluate;
c. contacting the second eluate with a MMC matrix such that the polypeptide binds to the MMC matrix, and selectively eluting the polypeptide from the MMC matrix in a third eluate; and
d. contacting the third eluate with a second AEX matrix under conditions such that the polypeptide binds to the second AEX matrix, and selectively eluting the polypeptide from the second AEX, thereby improving the serum half-life of the composition,
optionally wherein the polypeptide comprises: an amino acid sequence that is at least 85%, 90%, 95%, or 99% identical to SEQ ID NO: 1; or that comprises the amino acid sequence of SEQ ID NO: 1; and/or
the method further comprises adjusting the pH of the first eluate from step (a) to at least 10.5 and at most 11.5 before contacting the first eluate with the HIC matrix of step (b).

97-98. (canceled)

99. A composition comprising a plurality of polypeptides, each polypeptide of the plurality comprising the amino sequence of SEQ ID NO: 1 linked to one or more glycan species, wherein the one or more glycan species are linked to the polypeptide at one or more of amino acid positions N187, N206, and T212 of SEQ ID NO: 1.

100. The composition of claim 99, wherein:
1) the glycan species at amino acid position N187 of SEQ ID NO: 1 are selected from the group consisting of:
Hex5HexNAc4FucNeuAc2;
Hex6HexNAc5FucNeuAc2;
Hex5HexNAc4FucNeuAc;
Hex6HexNAc5FucNeuAc3;
Hex4HexNAc4FucNeuAc;
Hex5HexNAc5NeuAc2;
Hex5HexNAc4Fuc;
Hex3HexNAc4Fuc;
Hex4HexNAc4Fuc;
Hex6HexNAc5Fuc;
and
Hex5HexNAc5Fuc;
2) the glycan species at amino acid position N206 of SEQ ID NO: 1 are selected from the group consisting of:
Hex6HexNAc5FucNeuAc3;
Hex5HexNAc4FucNeuAc2;
Hex6HexNAc5FucNeuAc2;
Hex7HexNAc6FucNeuAc3;
Hex6HexNAc5FucNeuAc;
Hex5HexNAc4FucNeuAc;
Hex5HexNAc4Fuc;
and
Hex4HexNAc4Fuc;
and/or
3) the glycan species at amino acid position T212 of SEQ ID NO: 1 are selected from the group consisting of:
HexHexNAc;
HexHexNAcNeuAc;
and
HexHexNAcNeuAc2;
wherein Hex represents hexose, HexNAc represents N-acetylhexosamine, NeuAc represents N-acetylneuraminic acid, Fuc represents fucose, and the number represents the number of each glycan structure.

101-102. (canceled)

103. The composition of claim 99, wherein:
1) the overall percent of glycan species at amino acid position N187 of SEQ ID NO: 1 of the plurality of polypeptides in the composition comprises:
about 60% to about 70% Hex5HexNAc4FucNeuAc2;
about 4% to about 6% Hex6HexNAc5FucNeuAc2;
about 7% to about 10% Hex5HexNAc4FucNeuAc;
about 15% to about 17% Hex6HexNAc5FucNeuAc3; and
about 3% to about 4% Hex5HexNAc5NeuAc2;
2) the overall percent of glycan species at amino acid position N206 of SEQ ID NO: 1 of the plurality of polypeptides in the composition comprises:
about 3% to about 5% Hex6HexNAc5FucNeuAc3;
about 75% to about 85% Hex5HexNAc4FucNeuAc2;
about 2% to about 4% Hex6HexNAc5FucNeuAc2;
about 5% to about 12% Hex5HexNAc4FucNeuAc; and
about 1% to about 3% Hex5HexNAc4Fuc; and/or
3) the overall percent of glycan species at amino acid position T212 of SEQ ID NO: 1 of the plurality of polypeptides in the composition comprises:
about 14% to about 18% HexHexNAcNeuAc; and
about 8% to about 13% HexHexNAcNeuAc2;
wherein Hex represents hexose, HexNAc represents N-acetylhexosamine, NeuAc represents N-acetylneuraminic acid, Fuc represents fucose, and the number represents the number of each glycan structure.

104-108. (canceled)

109. A composition comprising a plurality of polypeptides, each polypeptide of the plurality comprising circularly permuted IL-2 fused to the extracellular portion of an IL-2Rα chain, wherein the composition comprises a capillary isoelectric focusing (cIEF) profile as depicted in FIG. 15.

110. The composition of claim 109, comprising a cIEF profile peak at one or more of about pI 5.73, about pI 5.93, about pI 6.09, about pI 6.28, about pI 6.38, about pI 6.48, about pI 6.53, about pI 6.66, about pI 6.82, and about pI 7.02, optionally:
comprising a peak area percent of:
about 8% to about 12% at pI 5.93;
about 18% to about 26% at pI 6.09;
about 22% to about 26% at pI 6.38; and
about 18% to about 28% at pI 6.66; or
comprising a peak area percent of:
about 1.5% to about 2.5% at pI 5.73;
about 8% to about 12% at pI 5.93;
about 18% to about 26% at pI 6.09;
about 3.5% to about 4.5% at pI 6.28;
about 22% to about 26% at pI 6.38;
about 3% to about 5% at pI 6.48;
about 4% to about 6% at pI 6.53;
about 18% to about 28% at pI 6.66;
about 2% to about 6% at pI 6.82; and
about 0% to about 3% at pI 7.02, optionally wherein:
the peak at about pI 5.73 comprises the combination of peaks at about pI 5.70 and about pI 5.76; and/or
the peak at about pI 5.93 comprises the combination of peaks at about pI 5.89 and about pI 5.97.

111-114. (canceled)

115. A composition comprising a plurality of polypeptides, each polypeptide of the plurality comprising the amino sequence of SEQ ID NO: 1 linked to one or more glycan species, wherein the one or more glycan species are linked to the polypeptide at one or more of amino acid positions N187, N206, and T212 of SEQ ID NO: 1, produced by the methods of claim 1.

116. A composition comprising a plurality of polypeptides, each polypeptide of the plurality comprising circularly permuted IL-2 fused to the extracellular portion of an IL-2Rα chain, wherein the composition comprises a capillary isoelectric focusing (cIEF) profile as depicted in FIG. 15, produced by the methods of claim 1.
